(19) United States
(12) Patent Application Publication
(10) Pub. No.: US 2025/0109387 A1
(43) Pub. Date: Apr. 3, 2025

(54) SMOOTH MUSCLE CELLS SEEDED IN MICROCHANNELS WITHIN HYDROGELS TO FORM FUNCTIONAL MEDIAL LAYERS IN ENGINEERED MICROVASCULATURE

(71) Applicant: VANDERBILT UNIVERSITY, Nashville, TN (US)

(72) Inventors: Leon M. Bellan, Nashville, TN (US); Victoria Vest, Nashville, TN (US); James D. West, Nashville, TN (US)

(21) Appl. No.: 18/897,291

(22) Filed: Sep. 26, 2024

Related U.S. Application Data
(60) Provisional application No. 63/597,785, filed on Nov. 10, 2023.
(60) Provisional application No. 63/586,209, filed on Sep. 28, 2023.

(51) Int. Cl. C12N 5/071

(52) U.S. Cl. CPC C12N 5/069; C12N 2500/05; C12N 2501/998; C12N 2501/999; C12N 2513/00; C12N 2533/90

(57) ABSTRACT

In one aspect, the disclosure relates to engineered microvessels including a microchannel formed in a hydrogel material; and a medial layer including a plurality of mural cells such as, for example, smooth muscle cells and/or pericytes; wherein each one of the plurality of mural cells is in contact with a wall of the microchannel; and wherein the plurality of mural cells are helically aligned at an angle relative to a lengthwise axis of the microchannel. The microvessels have narrow diameters consistent with resistance microvessels such as small resistance arteries and arterioles and tunable stiffness resulting from degree of crosslinking within the hydrogel material. Also disclosed are methods of making the microvessels, microvessel networks including the microvessels, and engineered organs and tissues including the microvessels. Furthermore, disclosed is a method for inducing vasoconstriction and vasodilation in the microvessels, networks, and engineered organs and tissues.

### CROSS REFERENCE TO RELATED APPLICATIONS

This application claims the benefit of U.S. Provisional Application No. 63/586,209 filed on Sep. 28, 2023, and U.S. Provisional Application No. 63/597,785 filed on Nov. 10, 2023, each of which is incorporated herein by reference in its entirety.

### STATEMENT REGARDING FEDERALLY SPONSORED RESEARCH OR DEVELOPMENT

This invention was made with government support under grant number 2050255 awarded by the National Science Foundation. The government has certain rights in the invention.

### BACKGROUND

Changing metabolic needs and environmental circumstances cause variation in the perfusion required to appropriately sustain various tissues and organs in the body. While overall blood flow can be modulated by the heart, perfusion is locally modulated by changes in vascular resistance caused by vascular smooth muscle cells (SMCs) in the medial layer acting to constrict or dilate the vessel lumen, ensuring appropriate local pressure-flow relationships (resistance). When vascular resistance is not appropriately regulated in vivo, a host of debilitating conditions can arise, including hypertension, orthostatic intolerance, myocardial ischemia, and brain dysfunction. Current tissue engineered microvasculature does not correctly mimic the coaxially-organized microanatomy, physiology, and molecular biology of resistance vessels (small resistance arteries and arterioles) and thus cannot regulate vascular resistance. Over the past decade, the field of tissue engineering has made significant progress towards creating microvasculature in tissue constructs for the purposes of overcoming diffusion limitations to maintain cells in thick scaffolds, with a wide range of approaches demonstrating the formation of lumen networks. The vast majority of these patterning techniques form channels that are in the arteriole (or larger) size range, with diameters greater than ˜50 microns. These channels, however, are then simply lined with endothelial cells (ECs) such that their microanatomy mimics that of capillaries. There is clearly a mismatch between the lumen size and microanatomy, resulting in structures that are either far too large to be called capillaries, or lacking helically-aligned smooth muscle cells to functionally mimic resistance vessels and enable regulation of vascular tone.

Tissue engineering approaches to form functional endothelium are mature. The selective permeability of the endothelium enables this barrier to moderate which soluble compounds pass between the blood and surrounding tissue. This occurs at the capillaries, the “exchange vessels”. All larger vessels (in the microvasculature, these can be referred to as “non-capillary microvessels”) serve not as exchange vessels but as either (for very large vessels) conduit vessels or (for vessels in the 20-400 μm range) resistance vessels that modulate local fluidic resistance by changing their diameter. The “actuators” that allow such vessels to change diameter are contractile phenotype SMCs, aligned helically around the vessel in the medial layer. This architecture is important because it connects contraction of the SMCs to constriction of the vessel. Resistance microvessels will have only 1-3 layers of SMCs in the medial layer, while larger vessels may have more layers.

There is clearly a gap in functionality of engineered microvasculature. Coaxing SMCs within a single microchannel in a hydrogel to form an appropriate helically-aligned architecture remains an unmet challenge. Achieving appropriate SMC architecture in perfusable branching microchannel networks is an even more challenging prospect, yet will be necessary for microvasculature with resistance vessel functionality. Given the importance of the medial layer in the function of resistance vessels, it is critical to establish approaches to form this microanatomical feature, with correct architecture and function, in engineered microvasculature.

To date, there have been few efforts devoted to engineering a functional medial layer in engineered microvasculature. Early efforts were able to place cells in large tubular volumes surrounding the lumen of an engineered vessel, but showed only viability, no contractile phenotype or appropriate architecture. Other early efforts have focused on larger vessels (mm/cm scale) and have shown promising results, but the fabrication approaches used for such tissue engineered vascular grafts do not translate to the microscale, and the medial layer thicknesses are not appropriate. Most recently, two groups have demonstrated 3D printing approaches to form hydrogel tubes containing SMCs, and demonstrated some initial level of contractile functionality. However, there was no demonstration of appropriate circumferential architecture, and the thicknesses of the medial layers formed were far larger than appropriate for the vessel produced. Finally, one recent study demonstrated self-assembled SMC-coated tubes with appropriate architecture, but no ability to form branching structures, support for perfusion, or integration into other cell-laden gels.

What is needed is a way of forming a medial layer in microvessels patterned in a hydrogel by introducing SMCs via flow into a channel (or channel network), allowing them to adhere, and giving them appropriate cues to a) align helically (i.e. circumferentially) and b) exhibit contractile phenotype (as opposed to synthetic phenotype, in which they do not contract but instead deposit matrix proteins). In an aspect, microvessels formed in this manner would enable the SMCs to serve as “actuators” in the medial layer such that they are able to constrict or dilate the vessel. In an aspect, after the SMCs have been introduced by flow and allowed to adhere to channel walls, they must be provided with an environment such that they exhibit the desired architecture and phenotype. These needs and other needs are satisfied by the present disclosure.

### SUMMARY

In accordance with the purpose(s) of the present disclosure, as embodied and broadly described herein, the disclosure, in one aspect, relates to engineered microvessels including a microchannel formed in a hydrogel material; and a medial layer including a plurality of mural cells such as, for example, smooth muscle cells and/or pericytes; wherein each one of the plurality of mural cells is in contact with a wall of the microchannel; and wherein the plurality of mural cells are helically aligned at an angle relative to a lengthwise axis of the microchannel. The microvessels have narrow diameters consistent with resistance microvessels such as small resistance arteries and arterioles and tunable stiffness resulting from degree of crosslinking within the hydrogel material. Also disclosed are methods of making the microvessels, microvessel networks including the microvessels, and engineered organs and tissues including the microvessels. Furthermore, disclosed is a method for inducing vasoconstriction and vasodilation in the microvessels, networks, and engineered organs and tissues.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows a 55 μm diameter channel formed in gelatin (crosslinked enzymatically using microbial transglutaminase [mTG]) by casting over a removable wire. Fluorescence signal is from a solution of 20 nm green fluorescent beads injected into the channel.

FIG. 2 shows RFP-expressing human aortic SMCs seeded in a channel formed in gelatin, without any coating on the channel walls, taken after static culture for 24 hours after seeding. This single image is a flattened confocal z-stack. Note the lack of circumferential organization. Scale bar is 250 μm.

FIGS. 3A-3I show human ductus arteriosus SMCs cultured in a hydrogel tube (6.4 mm OD, 2.4 mm ID) under various flow conditions (pulsatile [FIGS. 3A, 3D, and 3G], static [FIGS. 3B, 3E, and 3H], and constant flow [FIGS. 3C, 3F, and 3I]) for 7 days. FIGS. 3A-3C) Results from image analysis of maximum intensity projections of confocal imaging of SMCs stained for αSMA (scale bar 100 μm) wherein the cell orientation is color coded. FIGS. 3D-3F) Histograms indicating number of cells binned by orientation angle FIGS. 3G-3I) Characteristic flow waveforms for the 3 different culture conditions, measured with a Sensirion flow sensor.

FIG. 4 shows SMCs in a channel within gelatin (normal stiffness) with collagen IV and fibronectin coating for adhesion. Note the lack of any consistent orientation of the cells.

FIGS. 5A-5D show exemplary embodiments with (FIG. 5A) no ECM matrix protein coating on gelatin walls; (FIG. 5B) only collagen IV coating on gelatin walls (400 μg/mL); (FIG. 5C), collagen IV (400 μg/mL) and fibronectin (100 μg/mL) coating on gelatin walls; and (FIG. 5D) collagen (400 μg/mL), fibronectin (100 μg/mL), vitronectin (50 μg/mL) coating on channel walls.

FIGS. 6A-6F show representative images of HASMCs cultured on various ECM components. Contractile marker expression (Green: αSMA, Red: SM-MHC) in HASMCs seeded on (FIG. 6A) Fibronectin (125 μg/mL)+Collagen IV (125 g/mL), (FIG. 6B) Vitronectin (250 μg/mL), (FIG. 6C) Fibronectin (125 μg/mL)+Vitronectin (125 g/mL), (FIG. 6D) Tropoelastin (250 μg/mL), (FIG. 6E) Fibronectin (125 μg/mL)+Laminin (125 μg/mL), or (FIG. 6E) Vitronectin (125 μg/mL)+Laminin (125 μg/mL). Scale bar 50 μm.

FIG. 7 shows a summary of ECM effects on HASMC culture.

FIG. 8 shows morphological differences in RFP-expressing HASMCs cultured on substrates with various storage moduli of 8 kPa (left), 16 kPa (middle), and 32 kPa (right). ECM coating employed was collagen IV+fibronectin.

FIG. 9 shows GFP-EC and RFP-SMC layered co-culture in a 300 μm channel within mTG-gelatin.

FIG. 10 shows storage modulus of various formulations of gelatin crosslinked with 1% mTG.

FIG. 11 shows an illustration of cell seeding approach and timeline.

FIG. 12 shows confocal microscopy image of GFP-HUVEC (green) and helically aligned RFP-HASMC (red) layered co-culture in a microchannel within mTG-gelatin. Scale bar 200 μm. Inset: Cross sectional image showing intimal EC layer (interior, green) and medial SMC layer (exterior, red). Scale bar 100 μm.

FIGS. 13A-13D show cyclic stretch generated in a hydrogel through perfusion with a peristaltic pump. FIGS. 13A-13B: Fluorescence microscopy images showing lumen diameter change during a pulse. FIG. 13C: Stretch magnitude increases with increasing flow rate and FIG. 13D: decreases with increasing gelatin or crosslinker content.

FIG. 14 shows when exposed to pathophysiological flow, RFPHASMCs seeded on the microchannel wall migrate into the surrounding hydrogel.

FIG. 15 shows that using the disclosed pumps and a Millar catheter, varying pressure waveforms can be generated and measured to mimic different varieties of vessels. This data was measured in a 900 μm microchannel in mTG-gelatin.

FIGS. 16A-16F show HASMCs with GCaMP6f cultured in (FIGS. 16A-16C) 2D and (FIGS. 16D-16F) 3D. (FIGS. 16A, 16D) Fluorescence prior to ET-1 exposure. (FIGS. 16B, 16E) Fluorescence after exposure to ET-1. (FIGS. 16C, 16F) Field-of-view fluorescence over time.

FIG. 17A shows constructs are anastomosed to the ischiatic artery (green arrow) in a chicken thigh, which is then perfused with blue dye to demonstrate patency. Red square is field of view for (FIG. 17B). FIG. 17B shows prior to perfusion with blue dye, imaging of 200 nm (blue) and 2 μm (green) Fluospheres enables channel diameter (yellow line) and flow characterization, respectively. FIG. 17C shows results of PIV analysis.

FIG. 18 shows assembly of a mold for making a freestanding gelatin tube.

FIG. 19 shows a gelatin tube suspended in an otherwise empty PDMS mold.

FIG. 20 shows a ˜300 μm hollow channel with ˜100 μm gelatin walls.

FIG. 21 shows the elastic modulus of gelatin/mTG hydrogel slabs for different gelatin concentrations. Results shown are for experiments using one-time crosslinking with mTG.

FIG. 22 shows the elastic modulus of gelatin/mTG hydrogel slabs increases after a secondary exposure to mTG. Results shown are for experiments using a first, lower density crosslinking with mTG followed by secondary crosslinking with additional mTG after an initial crosslinking in order to enhance stiffness.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein is a method for forming a medial layer in microvessels formed in a hydrogel by introducing smooth muscle cells (SMCs) via flow into a channel or channel network, allowing them to adhere, and providing appropriate cues to align the SMCs helically (i.e. circumferentially) and induce the SMCs to exhibit contractile phenotype (as opposed to currently known synthetic phenotypes, in which the muscle cells do not contract but instead deposit matrix proteins). In an aspect, this enables the SMCs to serve as “actuators” in the medial layer such that they are able to constrict or dilate the microvessels. In one aspect, in current model systems, SMCs are often seeded in a “volume” instead of on a “surface,” which is not appropriate given the layering architecture expected for resistance vessels. In an aspect, the concept of introducing cells by flow and allowing them to adhere to form a monolayer has typically not been employed for SMCs.

In an aspect, after the SMCs have been introduced by flow, they must be provided with an environment such that they exhibit the desired architecture and phenotype. Further in this aspect, using the right combination of matrix proteins for an adhesion layer (applied prior to cell seeding), hydrogel stiffness, and post-seeding pulsatile flow, helical alignment close to what is observed in nature can be achieved. In a further aspect, SMC/endothelial cell (EC) co-cultures can then be formed by subsequently seeding ECs over the SMC layer. In one aspect, this is important because crosstalk between the ECs and SMCs is critical for correct regulation of vessel diameter. Further in this aspect, the ECs send various biochemical control signals to the SMCs.

### Microvessels

In one aspect, disclosed herein is a microvessel including at least:
(a) a microchannel formed in a hydrogel material; and
(b) a medial layer including a plurality of mural cells comprising smooth muscle cells, pericytes, or both smooth muscle cells and pericytes

In a further aspect, each one of the plurality of mural cells (e.g., smooth muscle cells, pericytes, or both, is in contact with a wall of the microchannel; and the plurality of mural cells are helically (i.e., circumferentially) aligned at an angle relative to a lengthwise axis of the microchannel, where a lengthwise axis refers to the flow direction in the microvessel. In one aspect, the angle can be from about 30 degrees to about 90 degrees, or about 30 degrees to about 60 degrees, about 60 degrees to about 90 degrees, about 40 degrees to about 80 degrees, or about 30, 35, 40, 45, 50, 55, 60, 65, 70, 75, 80, 85, or about 90 degrees, or a combination of any of the foregoing values, or a range encompassing any of the foregoing values. In one aspect, the 90 degree angle is perpendicular to direction of flow in the channel.

In another aspect, the plurality of mural cells can be derived from a mammal, such as, for example, human brain, coronary, pulmonary, renal, skeletal muscle, hepatic, mesenteric, retinal, pancreatic, umbilical artery, umbilical vein microvascular smooth muscle cells, or any combination thereof. In another aspect, the plurality of mural cells are arranged in a plurality of layers. In some aspects, the microvessels include a layer of coating material such as collagen, fibronectin, vitronectin, laminin, elastin, cyclic RGD peptide, or any combination thereof between each layer of the plurality, or between the first layer and the channel wall.

In any of these aspects, the microchannels have a lumen diameter of less than about 2000 μm, or of less than about 1000 μm, or of less than about 500 μm, or of less than about 100 μm, or of from about 10 μm to about 2000 μm, about 50 μm to about 1000 μm, about 50 μm to about 100 μm, or about 10 to about 50 μm or about 5, 25, 50, 100, 200, 300, 400, 500, 600, 700, 800, 900, 1000, 1250, 1500, 1750, or about 2000 μm, or a combination of any of the foregoing values, or a range encompassing any of the foregoing values.

In still another aspect, the microvessel can include a layer of endothelial cells lining the lumen of the microvessel. In one aspect, these can be human brain, coronary, pulmonary, renal, skeletal muscle, hepatic, mesenteric, retinal, pancreatic, umbilical artery, umbilical vein microvascular endothelial cells, any combination thereof, or another endothelial cell type.

In any of these aspects, the hydrogel can include modified or unmodified gelatin, collagen, alginate, agarose, gelatin methacrylate, Matrigel, fibrin, polyethylene glycol, elastin, cellulose, or any combination thereof. In one aspect, when the hydrogel is or includes gelatin, the gelatin can be crosslinked. In a further aspect, crosslinking can be accomplished enzymatically using microbial transglutaminase (mTG). In an aspect, the gelatin can be present at from about 0.5% (w/v) to about 20% (w/v), or from about 10% (w/v) to about 18% (w/v), or at about 15% (w/v) in the hydrogel, with the remaining volume made up of water, salts, enzymes, crosslinkers, and the like, and the mTG can be present in an amount of from about 0.1% to about 5% w/v, about 0.1% to about 4%, about 0.2% to about 3%, or at about 1% w/v in the hydrogel material.

In some aspects, the microvessels further include elastin. In a further aspect, the elastin can be present in an amount of from about 5% to about 50% w/w relative to the gelatin, about 5% to about 25%, about 5% to about 15%, or about 5% to about 10% w/w relative to the gelatin, or at about 5, 10, 15, 20, 25, 30, 35, 40, 45, or about 50% w/w relative to the gelatin.

In one aspect, the matrix in which the microchannel is formed can have a stiffness of from about 0.1 to about 150 kPa, or from about 0.1 to about 100 kPa, from about 10 to about 50 kPa, or from about 10 to about 30 kPa, or can have a stiffness of 0.1, 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, or about 150 kPa, or a combination of any of the foregoing values, or a range encompassing any of the foregoing values.

Also disclosed herein are microvessel networks including a plurality of interconnected microvessels, wherein the microvessels are described above. In some aspects, the plurality of interconnected microvessels form a branched structure. In a further aspect, the branched microvessels can have different diameters or the same diameter.

Furthermore, disclosed herein are engineered tissues or organs including the microvessel networks. In some aspects, the engineered tissue or organ can include neural tissue.

### Method for Inducing Vasoconstriction or Vasodilation in an Engineered Microvessel

The smooth muscle cells of the microvessels and microvessel networks described herein have a contractile phenotype and can be induced to vasoconstrict, vasodilate, or both. In one aspect, disclosed herein is a method for inducing vasoconstriction, vasodilation, or both in the disclosed microvessels, networks, tissues, and organs, the method including flowing a solution holding at least one stimulus molecule through the microvessel.

In one aspect, vasoconstriction can be induced and the at least one stimulus molecule can be endothelin-1 or U46619. In a further aspect, the endothelin-1 can be flowed through in a concentration of about 500 pM to about 500 nM, about 1 nM to about 500 nM, about 1 nM to about 250 nM, or about 500 or 750 pM or about 1, 25, 50, 75, 100, 125, 150, 175, 200, 225, 250, 275, 300, 325, 350, 375, 400, 425, 450, 475, or about 500 nM, or a combination of any of the foregoing values, or a range encompassing any of the foregoing values, in the solution.

In an alternative aspect, the U46619 can be flowed through in a concentration of about 30 nM to about 100 nM, or about 30 to about 70 nM, about 30 to about 50 nM, or about 50 to about 70 nM, or about 30, 35, 40, 45, 50, 55, 60, 65, 70, 75, 80, 85, 90, 95, or about 100 nM, or a combination of any of the foregoing values, or a range encompassing any of the foregoing values, in the solution.

In still another aspect, vasodilation can be induced and the at least one stimulus molecule can be HA-1077 or sodium nitroprusside. In some aspects, vasodilation can be induced on a microvessel that has previously been vasoconstricted. In a further aspect, the HA-1077 can be flowed through in a concentration of about 1 μM to about 100 μM, about 1 to about 75 UM, about 10 to about 50 μM, or about 25 to about 50 μM, or at about 1, 5, 10, 15, 20, 25, 30, 35, 40, 45, 50, 55, 60, 65, 70, 75, 80, 85, 90, 95, or about 100 UM in the solution. In an alternative aspect, the sodium nitroprusside can be flowed through in a concentration of about 0.01 UM to about 1 μM, about 0.01 to about 0.5 M, about 0.05 to about 0.5 μM, or about 0.25 to about 0.5 UM, or at about 0.01, 0.025, 0.05, 0.075, 0.1, 0.25, 0.5, 0.75, or about 1 μM, or a combination of any of the foregoing values, or a range encompassing any of the foregoing values, in the solution.

### Method for Making the Microvessels

In one aspect, disclosed herein is a method for making a microvessel including at least the following steps:
(a) fabricating a microchannel in a hydrogel material;
(b) seeding a plurality of mural cells, including smooth muscle cells, pericytes, or both into the microchannel, wherein the individual cells of the plurality adhere to a lumen wall of the microchannel;
(c) subjecting the plurality of mural cells to at least one treatment to align the cells helically, to cause the plurality of mural cells to exhibit a contractile phenotype, or both.

In one aspect, the matrix in which the microchannel is formed is seeded with a second type of cells by mixing the hydrogel with cells prior to gelation. In a further aspect, these cells may be fibroblasts, hepatocytes, islet cells, osteoblasts, induced pluripotent stem cells, neurons, glial cells, or another type of cell (or mixture of multiple cell types).

In a further aspect, the plurality of mural cells in step (b) can be seeded by flowing a suspension of cells through a lumen of the microchannel. In one aspect, before step (b), a coating material including collagen, fibronectin, vitronectin, laminin, elastin, cyclic RGD peptide, or any combination thereof can be flowed into the microchannel to coat the plurality of mural cells, and step (b) can be repeated one or more times prior to moving to step (c). In any of these aspects, a coating material can be the substrate for a first layer of cells of the plurality, and can be applied between layers when more than one layer of cells is present.

In one aspect, the at least one treatment can be application of pulsatile flow through the lumen of the microchannel. In a further aspect, the pulsatile flow can have a frequency of about 1 Hz and a flow velocity of from about 0 to about 5 cm/s, 0 to about 3 cm/s, 0 to about 1 cm/s, or of about 0 to 0.5, 0 to 0.25, 0.25 to 0.75, or 0.75 to about 1 cm/s, or can be 0, 0.1, 0.2, 0.3, 0.4, 0.5, 0.6, 0.7, 0.8, 0.9, 1, 1.5, 2, 2.5, 3, 3.5, 4, 4.5, or about 5 cm/s, or a combination of any of the foregoing values, or a range encompassing any of the foregoing values. Further in this aspect, pulsatile flow can be provided by a pump controlled by a microcontroller programmed to provide the pulsatile flow. In still another aspect, the at least one treatment can helically align the plurality of mural cells at an angle relative to a lengthwise axis of the microchannel.

In another aspect, the method can further include applying a plurality of endothelial cells to the lumen of the microvessel after performing step either step (b) or step (c).

In one aspect, the microchannel can be fabricated by bioprinting, microfabrication, stereolithography, removal of a sacrificial or removable template, or any combination thereof, prior to performing step (a). In a further aspect, the sacrificial template can be or include poly(N-isopropylacrylamide) (PNIPAM), polyvinyl caprolactam (PVCL), a poloxamer, carbohydrate glass, a wire, a filament, a glass capillary, a needle, or any combination thereof. In some aspects, the sacrificial template is a dissolvable polymeric substance such as, for example, polyvinyl caprolactam co-polymer.

In one aspect, the method can further include the step of applying a layer of endothelial cells to the lumen of the microvessel.

In any of these aspects, the hydrogel can include modified or unmodified gelatin, collagen, alginate, agarose, gelatin methacrylate, Matrigel, fibrin, polyethylene glycol, elastin, cellulose, or any combination thereof. In an aspect, the hydrogel can include gelatin, glycated collagen, gelatin methacrylate, alginate, or any combination thereof. In one aspect, the method further includes enzymatically crosslinking the hydrogel with microbial transglutaminase (mTG). In some aspects, the hydrogel material further includes elastin.

### Freestanding Microchannels

In one aspect, a tube (i.e., microchannel) can be formed by allowing a crosslinker (such as, for example, mTG) to diffuse radially out of a channel formed in a physically crosslinked gel (such as, for example, gelatin). Further in this aspect, the mTG or other crosslinker covalently crosslinks the gel in the cylindrical volume surrounding the channel. In another aspect, the physically crosslinked gel surrounding the covalently crosslinked gel is then liquified (such as, for example, by heating) and then washed away, allowing the freestanding microchannel to be removed. See FIGS. 18-20. Further information and parameters important to synthesis of the freestanding microchannels are presented below.

In one aspect, disclosed herein are freestanding microchannels that include a lumen and a continuous wall surrounding the lumen, where the wall is constructed from an enzymatically crosslinked hydrogel material. In some aspects, the freestanding microchannels further include a medial layer including a plurality of mural cells such as smooth muscle cells, pericytes, or both. Further in this aspect, each one of the plurality of mural cells is in contact with the interior wall of the microchannel and the plurality of mural cells are helically aligned at an angle relative to a lengthwise axis of the microchannel. In some aspects, the angle is from about 30 degrees to about 90 degrees, or is about 30, 35, 40, 45, 50, 55, 60, 65, 70, 75, 80, 85, or about 90 degrees, or a combination of any of the foregoing values, or a range encompassing any of the foregoing values.

In some aspects, the plurality of mural cells are derived from a mammal. In another aspect, when the plurality of mural cells are smooth muscle cells, they can be selected from human brain, coronary, pulmonary, renal, skeletal muscle, hepatic, mesenteric, retinal, pancreatic, umbilical artery, umbilical vein microvascular smooth muscle cells, or any combination thereof. In some aspects, the cells are arranged in a plurality of layers. In one aspect, the freestanding microchannel includes a layer of coating material as a substrate for the first layer of the plurality of mural cells and between each layer of the plurality. In a further aspect, the layer of coating material can be selected from collagen, fibronectin, vitronectin, laminin, elastin, cyclic RGD peptide, or any combination thereof.

In one aspect, the microchannel can have a lumen diameter of less than about 2000 μm, or of less than about 1500 μm, less than about 1000 μm, less than about 500 μm, or between about 10 μm to about 100 μm, such as, for example, 10, 15, 20, 25, 30, 35, 40, 45, 50, 55, 60, 65, 70, 75, 80, 85, 90, 95, or 100 μm.

In some aspects, the freestanding microchannel further includes a layer of endothelial cells lining the lumen of the microchannel. In another aspect, the endothelial cells can be human brain, coronary, pulmonary, renal, skeletal muscle, hepatic, mesenteric, retinal, pancreatic, umbilical artery, umbilical vein microvascular endothelial cells, or any combination thereof.

In any of these aspects, the hydrogel material can be selected from modified or unmodified gelatin, collagen, alginate, agarose, gelatin methacrylate, Matrigel, fibrin, polyethylene glycol, elastin, cellulose, or any combination thereof. In one aspect, the hydrogel material is gelatin. In another aspect, the enzymatically crosslinked hydrogel material has been crosslinked using microbial transglutaminase (mTG). In another aspect, the gelatin is present in an amount of from about 0.5% (w/v) to about 10% (w/v) in the hydrogel material, or is present at about 0.5, 1, 1.5, 2, 2.5, 3, 3.5, 4, 4.5, 5, 5.5, 6, 6.5, 7, 7.5, 8, 8.5, 9, 9.5, or about 10% (w/v), or is present at about 1% to about 10% or about 5% to about 10%. In another aspect, the mTG is present in an amount of from about 0.1% (w/v) to about 10% (w/v), or from about 5% (w/v) to about 10% (w/v) in the hydrogel material, or is present at about 0.1, 1, 1.5, 2, 2.5, 3, 3.5, 4, 4.5, 5, 5.5, 6, 6.5, 7, 7.5, 8, 8.5, 9, 9.5, or about 10%, or a combination of any of the foregoing values, or a range encompassing any of the foregoing values.

In another aspect, the matrix in which the microchannel is formed can have an elastic modulus of from about 0.1 kPa to about 150 kPa, or from about 1 kPa to about 25 kPa, or of about 0.1, 1, 5, 10, 15, 20, 25, 50, 75, 100, 125, or 150 kPa, or a combination of any of the foregoing values, or a range encompassing any of the foregoing values.

In one aspect, the matrix in which the microchannel is formed is seeded with cells by mixing the hydrogel with cells prior to gelation. These cells may be fibroblasts, hepatocytes, islet cells, osteoblasts, induced pluripotent stem cells, neurons, glial cells, or another type of cell (or mixture of multiple cell types).

Method for Making the Freestanding Microchannel and/or a Freestanding Microvessel

Also disclosed herein is a method for making the disclosed freestanding microchannel, the method including at least the steps of:
(a) fabricating a microchannel (or network of microchannels) in a physically crosslinked hydrogel material;
(b) contacting an inner surface of the microchannel with an amount of crosslinking agent sufficient to crosslink a radial thickness of the hydrogel material and wherein at least a portion of the physically crosslinked hydrogel material distal to a lumen of the microchannel does not become covalently crosslinked;
(c) heating the physically crosslinked hydrogel material to a temperature at or above its melting point to produce a melted hydrogel material; and
(d) removing the melted hydrogel material from the covalently crosslinked hydrogel material;
wherein the covalently crosslinked hydrogel material forms the freestanding microchannel.

In an aspect, a radial thickness of the hydrogel material of the freestanding microchannel (i.e., thickness of the wall of the microchannel) is from about 10 μm to about 250 μm, or from about 10 μm to about 150 μm, or is about 10, 25, 50, 75, 100, 125, 150, 175, 200, 225, or about 250 μm, or a combination of any of the foregoing values, or a range encompassing any of the foregoing values. In some aspects, the network of microchannels can have the same diameter or different diameters, including, but not limited to, arrangements such as smaller diameter channels branching off from a larger diameter main channel.

In another aspect, the crosslinking agent is mTG.

In one aspect, the hydrogel matrix in which the microchannel is formed is seeded with cells by mixing the hydrogel with cells prior to gelation. These cells may be fibroblasts, hepatocytes, islet cells, osteoblasts, induced pluripotent stem cells, neurons, glial cells, or another type of cell (or mixture of multiple cell types).

Also disclosed herein is a method for making a freestanding microvessel, the method including at least the steps of:
(a) seeding a plurality of mural cells comprising smooth muscle cells, pericytes, or both into the disclosed freestanding microchannel, wherein the smooth muscle cells adhere to a lumen wall of the microchannel; and
(b) subjecting the plurality of mural cells to at least one treatment to align the plurality of mural cells helically, to cause the plurality of mural cells to exhibit a contractile phenotype, or both.

In a further aspect, the mural cells can be seeded by flowing a suspension of smooth muscle cells through the lumen of the freestanding microchannel. In another aspect, before step (a) a coating material including collagen, fibronectin, vitronectin, laminin, elastin, cyclic RGD peptide, or any combination thereof is flowed into the microchannel to coat the plurality of mural cells and step (a) is repeated prior to moving to step (b). In another aspect, the at least one treatment is pulsatile flow through the lumen of the microchannel. In a further aspect, the pulsatile flow has a frequency of from about 0.1 Hz to about 10 Hz, or of about 0.1, 0.5, 1, 1.5, 2, 2.5, 3, 3.5, 4, 4.5, 5, 5.5, 6, 6.5, 7, 7.5, 8, 8.5, 9, 9.5, or about 10 Hz, or a combination of any of the foregoing values, or a range encompassing any of the foregoing values. In another aspect, the pulsatile flow has a flow velocity of from about 0 cm/s to about 5 cm/s, or of about 0, 0.5, 1, 1.5, 2, 2.5, 3, 3.5, 4, 4.5, or 5 cm/s, or a combination of any of the foregoing values, or a range encompassing any of the foregoing values. For example, in one aspect, the pulsatile flow can alternate between 0 cm/s and 5 cm/s or another positive flow velocity. In any of these aspects, the pulsatile flow can be provided by a pump controlled by a microcontroller programmed to provide pulsatile flow.

In an aspect, the at least one treatment helically aligns the plurality of mural cells at an angle relative to a lengthwise axis of the microchannel. In some aspects, the angle is from about 30 degrees to about 90 degrees, or is about 30, 35, 40, 45, 50, 55, 60, 65, 70, 75, 80, 85, or about 90 degrees, or a combination of any of the foregoing values, or a range encompassing any of the foregoing values.

In some aspects, the plurality of mural cells are derived from a mammal. In another aspect, when the plurality of mural cells are smooth muscle cells, they can be selected from human brain, coronary, pulmonary, renal, skeletal muscle, hepatic, mesenteric, retinal, pancreatic, umbilical artery, umbilical vein microvascular smooth muscle cells, or any combination thereof. In some aspects, the cells are arranged in a plurality of layers. In one aspect, the method for making the freestanding microchannel and/or microvessel includes applying a layer of coating material as a substrate for the first layer of the plurality of mural cells. In a further aspect, additional layers of cells, and the coating material can also be applied between each layer of the plurality. In a further aspect, the layer of coating material can be selected from collagen, fibronectin, vitronectin, laminin, elastin, cyclic RGD peptide, or any combination thereof.

In some aspects, the freestanding microchannel further includes applying a plurality of endothelial cells lining the lumen of the microchannel after performing either step (a) or step (b). In another aspect, the endothelial cells can be human brain, coronary, pulmonary, renal, skeletal muscle, hepatic, mesenteric, retinal, pancreatic, umbilical artery, umbilical vein microvascular endothelial cells, or any combination thereof.

In some aspects, the microchannel can be fabricated by bioprinting, microfabrication, stereolithography, removal of a sacrificial or removable template, or any combination thereof, during step (a). In another aspect, the sacrificial template can be made from poly(N-isopropylacrylamide) (PNIPAM), polyvinyl caprolactam (PVCL), a poloxamer, carbohydrate glass, a wire, a filament, a glass capillary, a needle, or any combination thereof. In some aspects, the sacrificial template is a dissolvable polymeric substance such as, for example, a polyvinyl caprolactam co-polymer.

In one aspect, the hydrogel includes gelatin, glycated collagen, gelatin methacrylate, alginate, or any combination thereof.

Method for Making a Higher Stiffness Microvessel within a Lower Stiffness Matrix

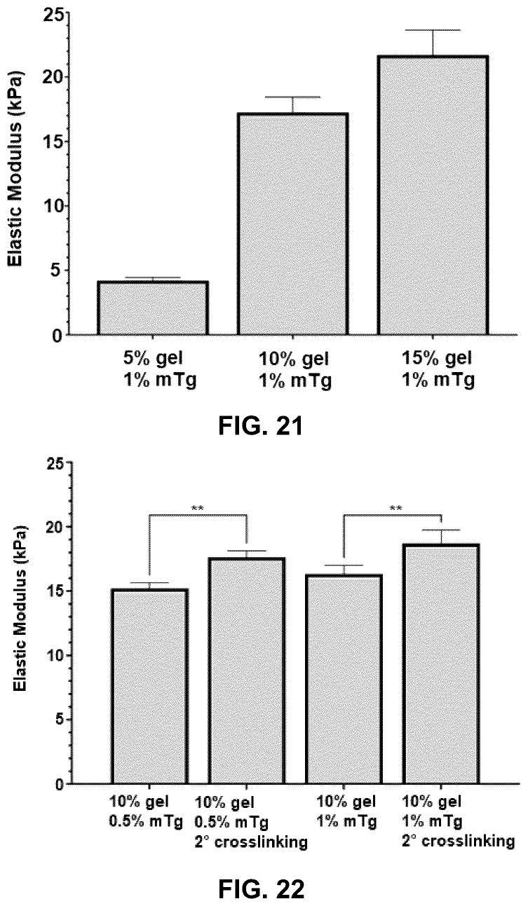

In another aspect, a tubular region of higher crosslinking density and thus higher stiffness can be formed surrounding a lumen formed in a covalently crosslinked gel. In one aspect, the process for making the stiff microchannel is similar to the method for making the freestanding microchannel, but instead of making the channel in a physically crosslinked gel, the channel would be made in a covalently crosslinked gel with relatively low crosslink density (i.e., a gel that is not fully crosslinked). FIG. 22 shows that secondary crosslinking can be performed after an initial crosslinking to enhance the stiffness (Young's modulus). FIG. 21 provides some baseline values for one-time crosslinking of different gel formulations. Further details about the procedure for making the stiff microchannel are provided below.

Also disclosed herein is a method for making a stiff microchannel in a bulk hydrogel medium, the method including at least the steps of:
(a) fabricating a microchannel (or network of microchannels) in a covalently crosslinked hydrogel material using a first amount of a crosslinker;
(b) contacting an inner surface of the microchannel with a second amount of crosslinking agent sufficient to secondarily crosslink a radial thickness of the hydrogel material at a greater density than a crosslinking density of the bulk hydrogel medium;
wherein a stiffness of the matrix adjacent to the microchannel that has been secondarily crosslinked is greater than a stiffness of the bulk hydrogel material.

In one aspect, the hydrogel matrix in which the microchannel is formed is seeded with cells by mixing the hydrogel with cells prior to gelation. These cells may be fibroblasts, hepatocytes, islet cells, osteoblasts, induced pluripotent stem cells, neurons, glial cells, or another type of cell (or mixture of multiple cell types).

In one aspect, the crosslinking agent can be mTG. In another aspect, the stiffness of the matrix directly surrounding the microchannel can be at least about 10% greater than the stiffness of the bulk hydrogel material. In yet another aspect, the method for making a stiff hydrogel further includes:
(c) seeding a plurality of mural cells including smooth muscle cells, pericytes, or both into the stiff microchannel, wherein the smooth muscle cells adhere to a lumen wall of the microchannel; and
(d) subjecting the plurality of mural cells to at least one treatment to align the plurality of mural cells helically, to cause the plurality of mural cells to exhibit a contractile phenotype, or both.

In one aspect, the mural cells are seeded by flowing a suspension of smooth muscle cells through a lumen of the stiff microchannel. In some aspects, before step (c), a coating material including collagen, fibronectin, vitronectin, laminin, elastin, cyclic RGD peptide, or any combination thereof is flowed into the microchannel to coat the plurality of mural cells and step (c) is repeated prior to moving to step (d).

In another aspect, the at least one treatment is pulsatile flow through the lumen of the microchannel. In a further aspect, the pulsatile flow has a frequency of from about 0.1 Hz to about 10 Hz, or of about 0.1, 0.5, 1, 1.5, 2, 2.5, 3, 3.5, 4, 4.5, 5, 5.5, 6, 6.5, 7, 7.5, 8, 8.5, 9, 9.5, or about 10 Hz, or a combination of any of the foregoing values, or a range encompassing any of the foregoing values. In another aspect, the pulsatile flow has a flow velocity of from about 0 cm/s to about 5 cm/s, or of about 0, 0.5, 1, 1.5, 2, 2.5, 3, 3.5, 4, 4.5, or 5 cm/s, or a combination of any of the foregoing values, or a range encompassing any of the foregoing values. For example, in one aspect, the pulsatile flow can alternate between 0 cm/s and 5 cm/s or another positive flow velocity. In any of these aspects, the pulsatile flow can be provided by a pump controlled by a microcontroller programmed to provide pulsatile flow.

In an aspect, the at least one treatment helically aligns the plurality of mural cells at an angle relative to a lengthwise axis of the stiff microchannel. In some aspects, the angle is from about 30 degrees to about 90 degrees, or is about 30, 35, 40, 45, 50, 55, 60, 65, 70, 75, 80, 85, or about 90 degrees, or a combination of any of the foregoing values, or a range encompassing any of the foregoing values.

In some aspects, the plurality of mural cells are derived from a mammal. In another aspect, when the plurality of mural cells are smooth muscle cells, they can be selected from human brain, coronary, pulmonary, renal, skeletal muscle, hepatic, mesenteric, retinal, pancreatic, umbilical artery, umbilical vein microvascular smooth muscle cells, or any combination thereof. In some aspects, the cells are arranged in a plurality of layers. In one aspect, the method for making the stiff microchannel and/or microvessel includes applying a layer of coating material as a substrate for the first layer of the plurality of mural cells. In a further aspect, additional layers of cells, and the coating material can also be applied between each layer of the plurality. In a further aspect, the layer of coating material can be selected from collagen, fibronectin, vitronectin, laminin, elastin, cyclic RGD peptide, or any combination thereof.

In some aspects, the method for making the stiff microchannel further includes applying a plurality of endothelial cells lining the lumen of the stiff microchannel after performing step (c). In another aspect, the endothelial cells can be human brain, coronary, pulmonary, renal, skeletal muscle, hepatic, mesenteric, retinal, pancreatic, umbilical artery, umbilical vein microvascular endothelial cells, or any combination thereof.

In some aspects, the microchannel can be fabricated by bioprinting, microfabrication, stereolithography, removal of a sacrificial or removable template, or any combination thereof, during step (a). In another aspect, the sacrificial template can be made from poly(N-isopropylacrylamide) (PNIPAM), polyvinyl caprolactam (PVCL), a poloxamer, carbohydrate glass, a wire, a filament, a glass capillary, a needle, or any combination thereof. In some aspects, the sacrificial template is a dissolvable polymeric substance such as, for example, a polyvinyl caprolactam co-polymer.

In one aspect, the hydrogel includes gelatin, glycated collagen, gelatin methacrylate, alginate, or any combination thereof.

Also disclosed herein are stiff microchannels made by the disclosed methods.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a coating material,” “a crosslinker,” or “a type of smooth muscle cell,” include, but are not limited to, mixtures or combinations of two or more such coating materials, crosslinkers, or types of smooth muscle cells, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a crosslinker refers to an amount that is sufficient to achieve the desired improvement in the property modulated by the formulation component, e.g. achieving the desired level of hydrogel stiffness. The specific level in terms of wt % in a composition required as an effective amount will depend upon a variety of factors including the amount and type of hydrogel material, number of layers of smooth muscle cells, organ or tissue type being modeled, and desired properties (contractility, stiffness, and the like) of the microvessel formed using the hydrogel material.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered aspects, which should not be confused with the claims.

Aspect 1. A microvessel comprising:
(a) a microchannel formed in a hydrogel material; and
(b) a medial layer including a plurality of mural cells comprising smooth muscle cells, pericytes, or both smooth muscle cells and pericytes;
wherein each one of the plurality of mural cells is in contact with a wall of the microchannel; and
wherein the plurality of mural cells are helically aligned at an angle relative to a lengthwise axis of the microchannel.

Aspect 2. The microvessel of aspect 1, wherein the hydrogel material is seeded with a second type of cells prior to gelation by mixing the cells with the hydrogel material.

Aspect 3. The microvessel of aspect 2, wherein the second type of cells comprise fibroblasts, hepatocytes, islet cells, osteoblasts, induced pluripotent stem cells, neurons, glial cells, another type of cell, or any combination thereof.

Aspect 4. The microvessel of any one of aspects 1-3, wherein the angle is from about 30 degrees to about 90 degrees.

Aspect 5. The microvessel of any one of aspects 1-4, wherein the plurality of mural cells comprise cells derived from a mammal.

Aspect 6. The microvessel of aspect 5, wherein the plurality of smooth muscle cells are human brain, coronary, pulmonary, renal, skeletal muscle, hepatic, mesenteric, retinal, pancreatic, umbilical artery, umbilical vein microvascular smooth muscle cells, or any combination thereof.

Aspect 7. The microvessel of any one of aspects 1-6, wherein the plurality of mural cells are arranged in a plurality of layers.

Aspect 8. The microvessel of any one of aspects 1-7, further comprising a layer of coating material as a substrate for a first layer mural cells, a first layer of the plurality, and between each layer of the plurality, when the plurality of layers is present.

Aspect 9. The microvessel of aspect 8, wherein the layer of coating material comprises collagen, fibronectin, vitronectin, laminin, elastin, cyclic RGD peptide, or any combination thereof.

Aspect 10. The microvessel of any one of aspects 1-9, wherein the microchannel has a lumen diameter of less than about 2000 μm.

Aspect 11. The microvessel of aspect 10, wherein the microchannel has a lumen diameter of from about 10 μm to about 50 μm.

Aspect 12. The microvessel of any one of aspects 1-10, further comprising a layer of endothelial cells lining a lumen of the microvessel.

Aspect 13. The microvessel of aspect 12, wherein the endothelial cells comprise human brain, coronary, pulmonary, renal, skeletal muscle, hepatic, mesenteric, retinal, pancreatic, umbilical artery, umbilical vein microvascular endothelial cells, or any combination thereof.

Aspect 14. The microvessel of any one of aspects 1-13, wherein the hydrogel comprises modified or unmodified gelatin, collagen, alginate, agarose, gelatin methacrylate, Matrigel, fibrin, polyethylene glycol, elastin, cellulose, or any combination thereof.

Aspect 15. The microvessel of aspect 14, wherein the gelatin is crosslinked.

Aspect 16. The microvessel of aspect 15, wherein the gelatin is crosslinked enzymatically with microbial transglutaminase (mTG).

Aspect 17. The microvessel of any one of aspects 14-16, wherein the gelatin is present at from about 0.5% to about 20% w/v in the hydrogel.

Aspect 18. The microvessel of aspect 16 or 17, wherein the mTG is present in an amount of from about 0.1% to about 10% w/v in the hydrogel material.

Aspect 19. The microvessel of any one of aspects 14-18, further comprising elastin.

Aspect 20. The microvessel of aspect 19, wherein the elastin is present at from about 5% to about 50% w/w relative to the gelatin.

Aspect 21. The microvessel of any one of aspects 1-20, wherein the microvessel has a stiffness of from about 0.1 to about 150 kPa.

Aspect 22. A microvessel network comprising a plurality of interconnected microvessels according to any one of aspects 1-21.

Aspect 23. An engineered tissue or organ comprising the microvessel network of aspect 22.

Aspect 24. The engineered tissue or organ of aspect 23, wherein the engineered tissue or organ comprises neural tissue.

Aspect 25. A method for inducing vasoconstriction, vasodilation, or both in the microvessel of aspect 1-21, the microvessel network of aspect 22, or the engineered tissue or organ of aspect 23 or 24, the method comprising flowing a solution comprising at least one stimulus molecule through the microvessel.

Aspect 26. The method of aspect 25, wherein the vasoconstriction is induced and wherein the at least one stimulus molecule comprises endothelin-1 or U46619.

Aspect 27. The method of aspect 26, wherein the endothelin-1 is flowed through in a concentration of about 500 pM to about 500 nM in the solution.

Aspect 28. The method of aspect 26, wherein the U46619 is flowed through in a concentration of about 30 nM to about 100 nM in the solution.

Aspect 29. The method of aspect 25, wherein vasodilation is induced and wherein the at least one stimulus molecule comprises HA-1077 or sodium nitroprusside.

Aspect 30. The method of aspect 25, wherein vasodilation is induced on a microvessel that has previously been vasoconstricted.

Aspect 31. The method of aspect 29 or 30, wherein the HA-1077 is flowed through in a concentration of about 1 μM to about 100 UM in the solution.

Aspect 32. The method of aspect 29 or 30, wherein the sodium nitroprusside is flowed through in a concentration of about 0.01 UM to about 1 μM in the solution.

Aspect 33. The method of aspect any one of aspects 25-32, wherein the solution is flowed through the microvessel at about 15 μL/min.

Aspect 34. A method for making a microvessel, the method comprising:
(a) fabricating a microchannel in a hydrogel material;
(b) seeding a plurality of mural cells comprising smooth muscle cells, pericytes, or both into the microchannel, wherein the smooth muscle cells adhere to a lumen wall of the microchannel;
(c) subjecting the plurality of mural cells to at least one treatment to align the plurality of mural cells helically, to cause the plurality of mural cells to exhibit a contractile phenotype, or both.

Aspect 35. The method of aspect 34, wherein the plurality of mural cells are seeded by flowing a suspension of smooth muscle cells through a lumen of the microchannel.

Aspect 36. The method of aspect 34 or 35, wherein before step (b), a coating material comprising collagen, fibronectin, vitronectin, laminin, elastin, cyclic RGD peptide, or any combination thereof is flowed into the microchannel to coat the plurality of mural cells and step (b) is repeated prior to moving to step (c).

Aspect 37. The method of any one of aspects 34-36, wherein the at least one treatment comprises application of pulsatile flow through the lumen of the microchannel.

Aspect 38. The method of aspect 37, wherein the pulsatile flow has a frequency of from about 0.1 Hz to about 10 Hz.

Aspect 39. The method of aspect 37 or 38, wherein the pulsatile flow has a flow velocity of from about 0 to about 5 cm/s.

Aspect 40. The method of any one of aspects 34-39, wherein the at least one treatment helically aligns the plurality of mural cells at an angle relative to a lengthwise axis of the microchannel.

Aspect 41. The method of aspect 40, wherein the angle is from about 30 degrees to about 90 degrees.

Aspect 42. The method of aspect any one of aspects 34-41, wherein the plurality of mural cells comprise cells derived from a mammal.

Aspect 43. The method of aspect 42, wherein the plurality of smooth muscle cells are human brain, coronary, pulmonary, renal, skeletal muscle, hepatic, mesenteric, retinal, pancreatic, umbilical artery, umbilical vein microvascular smooth muscle cells, or any combination thereof.

Aspect 44. The method of aspect any one of aspects 34-43, further comprising applying a first layer of coating material under the plurality of mural cells.

Aspect 45. The method of aspect 44, wherein the layer of coating material comprises collagen, fibronectin, vitronectin, laminin, elastin, cyclic RGD peptide, or any combination thereof.

Aspect 46. The method of aspect 44 or 45, further comprising applying one or more additional layers of smooth muscle cells, pericytes, coating material, or both, on the first layer of coating material.

Aspect 47. The method of any one of aspects 34-46, further comprising:
(d) applying a plurality of endothelial cells to a lumen of the microvessel after performing step (b) or step (c).

Aspect 48. The method of any one of aspects 34-47, further comprising fabricating the microchannel by bioprinting, microfabrication, stereolithography, removal of a sacrificial or removable template, or any combination thereof, prior to performing step (a).

Aspect 49. The method of aspect 48, wherein the sacrificial template comprises poly(N-isopropylacrylamide) (PNIPAM), polyvinyl caprolactam (PVCL), a poloxamer, carbohydrate glass, a wire, a filament, a glass capillary, a needle, or any combination thereof.

Aspect 50. The method of aspect 48 or 49, wherein the sacrificial template is a dissolvable polymeric substance.

Aspect 51. The method of aspect 50, wherein the dissolvable polymeric substance comprises a polyvinyl caprolactam co-polymer.

Aspect 52. The method of any one of aspects 34-51, further comprising applying a layer of endothelial cells to a lumen of the microvessel.

Aspect 53. The method of aspect 52, wherein the endothelial cells comprise human brain, coronary, pulmonary, renal, skeletal muscle, hepatic, mesenteric, retinal, pancreatic, umbilical artery, umbilical vein microvascular endothelial cells, or any combination thereof.

Aspect 54. The method of any one of aspects 34-53, wherein the hydrogel comprises gelatin, glycated collagen, gelatin methacrylate, alginate, or any combination thereof.

Aspect 55. The method of aspect 54, further comprising crosslinking the gelatin.

Aspect 56. The method of aspect 55, wherein crosslinking is accomplished enzymatically with microbial transglutaminase (mTG).

Aspect 57. The method of any one of aspects 54-56, wherein the gelatin is present at about 0.5% to about 20% w/v in the hydrogel material.

Aspect 58. The method of aspect 56 or 57, wherein the mTG is present in an amount of about 0.1% w/v to about 10% w/v in the hydrogel material.

Aspect 59. The method of any one of aspects 34-58, wherein the hydrogel material further comprises elastin.

Aspect 60. The method of aspect 59, wherein the elastin is present at from about 5% to about 50% w/w relative to the gelatin.

Aspect 61. A freestanding microchannel comprising a lumen and a continuous wall surrounding the lumen, wherein the wall comprises an covalently crosslinked hydrogel material.

Aspect 62. The freestanding microchannel of aspect 61, wherein the microchannel further comprises a medial layer including a plurality of mural cells comprising smooth muscle cells, pericytes, or both smooth muscle cells and pericytes;
wherein each one of the plurality of mural cells is in contact with the wall of the microchannel; and
wherein the plurality of mural cells are helically aligned at an angle relative to a lengthwise axis of the microchannel.

Aspect 63. The freestanding microchannel of aspect 62, wherein the angle is from about 30 degrees to about 90 degrees.

Aspect 64. The freestanding microchannel of aspect 62 or 63, wherein the plurality of mural cells comprise cells derived from a mammal.

Aspect 65. The freestanding microchannel of any one of aspects 62-64, wherein the plurality of smooth muscle cells are human brain, coronary, pulmonary, renal, skeletal muscle, hepatic, mesenteric, retinal, pancreatic, umbilical artery, umbilical vein microvascular smooth muscle cells, or any combination thereof.

Aspect 66. The freestanding microchannel of any one of aspects 62-65, wherein the plurality of mural cells are arranged in a plurality of layers.

Aspect 67. The freestanding microchannel of any one of aspects 62-66, further comprising a layer of coating material as a substrate for a first layer mural cells, a first layer of the plurality, and between each layer of the plurality, when the plurality of layers is present.

Aspect 68. The freestanding microchannel of aspect 67, wherein the layer of coating material comprises collagen, fibronectin, vitronectin, laminin, elastin, cyclic RGD peptide, or any combination thereof.

Aspect 69. The freestanding microchannel of any one of aspects 61-68, wherein the microchannel has a lumen diameter of less than about 2000 μm.

Aspect 70. The freestanding microchannel of aspect 69, wherein the microchannel has a lumen diameter of from about 10 μm to about 100 μm.

Aspect 71. The freestanding microchannel of any one of aspects 62-70, further comprising a layer of endothelial cells lining a lumen of the microchannel.

Aspect 72. The freestanding microchannel of aspect 71, wherein the endothelial cells comprise human brain, coronary, pulmonary, renal, skeletal muscle, hepatic, mesenteric, retinal, pancreatic, umbilical artery, umbilical vein microvascular endothelial cells, or any combination thereof.

Aspect 73. The freestanding microchannel of any one of aspects 61-72, wherein the hydrogel material comprises modified or unmodified gelatin, collagen, alginate, agarose, gelatin methacrylate, Matrigel, fibrin, polyethylene glycol, elastin, cellulose, or any combination thereof.

Aspect 74. The freestanding microchannel of aspect 73, wherein the hydrogel material comprises gelatin.

Aspect 75. The freestanding microchannel of any one of aspects 61-74, wherein the covalently crosslinked hydrogel material has been crosslinked using microbial transglutaminase (mTG).

Aspect 76. The freestanding microchannel of aspect 74 or 75, wherein the gelatin is present in an amount of from about 0.5% (w/v) to about 20% (w/v) in the hydrogel material.

Aspect 77. The freestanding microchannel of aspect 75 or 76, wherein the mTG is present in an amount of from about 0.01% (w/v) to about 10% (w/v) in the hydrogel material.

Aspect 78. The freestanding microchannel of any one of aspects 61-77, wherein the microchannel has an elastic modulus of from about 0.1 kPa to about 150 kPa.

Aspect 79. The freestanding microchannel of aspect 78, wherein the microchannel has an elastic modulus of from about 1 kPa to about 25 kPa.

Aspect 80. A method for making a freestanding microchannel, the method comprising:
(a) fabricating a microchannel in a physically crosslinked hydrogel material;
(b) contacting an inner surface of the microchannel with an amount of crosslinking agent sufficient to covalently crosslink a radial thickness of the hydrogel material and wherein at least a portion of the physically crosslinked hydrogel material distal to a lumen of the microchannel does not become covalently crosslinked;
(c) heating the physically crosslinked hydrogel material to a temperature at or above its melting point to produce a melted hydrogel material; and
(d) removing the melted hydrogel material from the covalently crosslinked hydrogel material;
wherein the covalently crosslinked hydrogel material forms the freestanding microchannel.

Aspect 81. The method of aspect 80, wherein the radial thickness of the hydrogel material is from about 50 μm to about 250 μm.

Aspect 82. The method of aspect 80 or 81, wherein the crosslinking agent comprises mTG.

Aspect 83. A method for making a freestanding microvessel, the method comprising:
(a) seeding a plurality of mural cells comprising smooth muscle cells, pericytes, or both into the freestanding microchannel of any one of aspects 80-82, wherein the smooth muscle cells adhere to a lumen wall of the microchannel; and
(b) subjecting the plurality of mural cells to at least one treatment to align the plurality of mural cells helically, to cause the plurality of mural cells to exhibit a contractile phenotype, or both.

Aspect 84. The method of aspect 83, wherein the plurality of mural cells are seeded by flowing a suspension of smooth muscle cells through a lumen of the freestanding microchannel.

Aspect 85. The method of aspect 83 or 84, wherein before step (a), a coating material comprising collagen, fibronectin, vitronectin, cyclic RGD peptide, or any combination thereof is flowed into the microchannel to coat the plurality of mural cells and step (a) is repeated prior to moving to step (b).

Aspect 86. The method of any one of aspects 83-85, wherein the at least one treatment comprises application of pulsatile flow through the lumen of the microchannel.

Aspect 87. The method of aspect 86, wherein the pulsatile flow has a frequency of from about 0.1 Hz to about 10 Hz.

Aspect 88. The method of aspect 86 or 87, wherein the pulsatile flow has a flow velocity of from about 0 to about 5 cm/s.

Aspect 90. The method of any one of aspects 83-89, wherein the at least one treatment helically aligns the plurality of mural cells at an angle relative to a lengthwise axis of the microchannel.

Aspect 91. The method of aspect 90, wherein the angle is from about 30 degrees to about 90 degrees.

Aspect 92. The method of any one of aspects 83-91, wherein the plurality of mural cells comprise cells derived from a mammal.

Aspect 93. The method of aspect 92, wherein the plurality of smooth muscle cells are human brain, coronary, pulmonary, renal, skeletal muscle, hepatic, mesenteric, retinal, pancreatic, umbilical artery, umbilical vein microvascular smooth muscle cells, or any combination thereof.

Aspect 94. The method of any one of aspects 83-93, further comprising applying a first layer of coating material under the plurality of mural cells.

Aspect 95. The method of aspect 94, wherein the layer of coating material comprises collagen, fibronectin, vitronectin, laminin, elastin, cyclic RGD peptide, or any combination thereof.

Aspect 96. The method of aspect 94 or 95, further comprising applying one or more additional layers of smooth muscle cells, pericytes, coating material, or both, on the first layer of coating material.

Aspect 97. The method of any one of aspects 83-96, further comprising:
(c) applying a plurality of endothelial cells to a lumen of the microvessel after performing step (a) or step (b).

Aspect 98. The method of aspect 97, wherein the endothelial cells comprise human brain, coronary, pulmonary, renal, skeletal muscle, hepatic, mesenteric, retinal, pancreatic, umbilical artery, umbilical vein microvascular endothelial cells, or any combination thereof.

Aspect 99. The method of any one of aspects 83-98, further comprising fabricating the microchannel by bioprinting, microfabrication, stereolithography, removal of a sacrificial or removable template, or any combination thereof, during step (a).

Aspect 100. The method of aspect 99, wherein the sacrificial template comprises poly(N-isopropylacrylamide) (PNIPAM), polyvinyl caprolactam (PVCL), a poloxamer, carbohydrate glass, a wire, a filament, a glass capillary, a needle, or any combination thereof.

Aspect 101. The method of aspect 99 or 100, wherein the sacrificial template is a dissolvable polymeric substance.

Aspect 102. The method of aspect 101, wherein the dissolvable polymeric substance comprises a polyvinyl caprolactam co-polymer.

Aspect 104. The method of any one of aspects 83-103, wherein the hydrogel comprises gelatin, glycated collagen, gelatin methacrylate, alginate, or any combination thereof.

Aspect 105. A method for making a stiff microchannel in a bulk hydrogel medium, the method comprising:
(a) fabricating a microchannel in a hydrogel material;
(b) crosslinking the hydrogel material using a first amount of a crosslinker; and
(c) contacting an inner surface of the microchannel with a second amount of crosslinking agent sufficient to secondarily covalently crosslink a radial thickness of the hydrogel material at a greater density than a crosslinking density of the bulk hydrogel medium;
wherein a stiffness of the microchannel that has been secondarily covalently crosslinked is greater than a stiffness of the bulk hydrogel material.

Aspect 106. The method of aspect 105, wherein the crosslinking agent comprises mTG.

Aspect 107. The method of aspect 105 or 106, wherein the stiffness of the microchannel is at least about 10% greater than the stiffness of the bulk hydrogel material.

Aspect 108. The method of any one of aspects 105-107, further comprising:
(d) seeding a plurality of mural cells comprising smooth muscle cells, pericytes, or both into the stiff microchannel, wherein the smooth muscle cells adhere to a lumen wall of the microchannel; and
(e) subjecting the plurality of mural cells to at least one treatment to align the plurality of mural cells helically, to cause the plurality of mural cells to exhibit a contractile phenotype, or both.

Aspect 109. The method of aspect 108, wherein the plurality of mural cells are seeded by flowing a suspension of smooth muscle cells through a lumen of the stiff microchannel.

Aspect 110. The method of aspect 108 or 109, wherein before step (d), a coating material comprising collagen, fibronectin, vitronectin, cyclic RGD peptide or any combination thereof is flowed into the microchannel to coat the plurality of mural cells and step (d) is repeated prior to moving to step (e).

Aspect 111. The method of any one of aspects 108-110, wherein the at least one treatment comprises application of pulsatile flow through the lumen of the microchannel.

Aspect 112. The method of aspect 111, wherein the pulsatile flow has a frequency of from about 0.1 Hz to about 10 Hz.

Aspect 113. The method of aspect 111 or 112, wherein the pulsatile flow has a flow velocity of from about 0 to about 5 cm/s.

Aspect 114. The method of any one of aspects 108-113, wherein the at least one treatment helically aligns the plurality of mural cells at an angle relative to a lengthwise axis of the stiff microchannel.

Aspect 115. The method of aspect 114, wherein the angle is from about 30 degrees to about 90 degrees.

Aspect 116. The method of any one of aspects 108-115, wherein the plurality of mural cells comprise cells derived from a mammal.

Aspect 117. The method of aspect 116, wherein the plurality of smooth muscle cells are human brain, coronary, pulmonary, renal, skeletal muscle, hepatic, mesenteric, retinal, pancreatic, umbilical artery, umbilical vein microvascular smooth muscle cells, or any combination thereof.

Aspect 118. The method of any one of aspects 108-117, further comprising applying a first layer of coating material under the plurality of mural cells.

Aspect 119. The method of aspect 118, wherein the layer of coating material comprises collagen, fibronectin, vitronectin, laminin, elastin, cyclic RGD peptide or any combination thereof.

Aspect 120. The method of aspect 118 or 119, further comprising applying one or more additional layers of smooth muscle cells, pericytes, coating material, or both, on the first layer of coating material.

Aspect 121. The method of any one of aspects 108-120, further comprising:
(f) applying a plurality of endothelial cells to a lumen of the microvessel after performing step (d) or step (e).

Aspect 122. The method of aspect 121, wherein the endothelial cells comprise human brain, coronary, pulmonary, renal, skeletal muscle, hepatic, mesenteric, retinal, pancreatic, umbilical artery, umbilical vein microvascular endothelial cells, or any combination thereof.

Aspect 123. The method of any one of aspects 105-122, further comprising fabricating the stiff microchannel by bioprinting, microfabrication, stereolithography, removal of a sacrificial or removable template, or any combination thereof, during step (a).

Aspect 124. The method of aspect 123, wherein the sacrificial template comprises poly(N-isopropylacrylamide) (PNIPAM), polyvinyl caprolactam (PVCL), a poloxamer, carbohydrate glass, a wire, a filament, a glass capillary, a needle, or any combination thereof.

Aspect 125. The method of aspect 123 or 124, wherein the sacrificial template is a dissolvable polymeric substance.

Aspect 126. The method of aspect 125, wherein the dissolvable polymeric substance comprises a polyvinyl caprolactam co-polymer.

Aspect 127. The method of any one of aspects 105-127, wherein the hydrogel comprises gelatin, glycated collagen, gelatin methacrylate, alginate, or any combination thereof.

Aspect 128. A stiff microchannel made by the method of any one of aspects 105-128.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Microvasculature Engineering Approach

The approach described herein is predicated on understanding how mechanical cues can be used to drive vascular smooth muscle cells (SMCs) in engineered microcirculatory networks to exhibit contractile phenotype, helically align, and respond to vasoactive stimuli by modulating vessel diameter (tone) and thereby regulate vascular resistance. When the ability to regulate microvascular resistance is impaired in vivo, a host of debilitating conditions can arise, including hypertension, orthostatic intolerance, complex regional pain syndrome, myocardial ischemia, brain dysfunction, and organ dysfunction and failure during sepsis. This highlights the crucial role of vascular resistance regulation (which occurs primarily in small resistance arteries and arterioles in the microvasculature) in maintaining homeostasis and healthy function throughout the body. To date, however, engineered microvasculature is incapable of regulating vascular resistance due to the lack of a functional medial layer containing correctly organized contractile phenotype SMCs. Future clinical use of engineered tissues without this capability is likely to lead to the debilitating conditions observed when vascular regulation is impaired. Scientific studies employing engineered tissues lacking this crucial feature may yield results inconsistent with the behavior of healthy natural tissue. The past decade has seen a myriad of techniques developed to fabricate microvascular networks in cell-laden gels for the purpose of maintaining cell viability throughout thick volumes of engineered tissue. While the field has made impressive progress towards forming endothelialized channel networks, natural vessels with diameters similar to those typically patterned (>50 μm lumen) are not simple capillaries (which have walls a single endothelial cell [EC] in thickness), but small resistance arteries, veins, arterioles and venules, with coaxial wall layers consisting of ECs (intima), vascular SMCs (media), and fibroblasts (adventitia). While there has been significant focus on endothelialization (and subsequent characterization and optimization of endothelium barrier properties), the lack of a functional medial layer of SMCs in patterned microvessel networks has yet to be addressed. Simply put, this means that microcirculatory systems resulting from current approaches are generally incapable of performing one of the crucial functions of the natural microcirculation: regulation of vascular resistance. To enable engineering processes that result in microvasculature capable of mimicking the vascular resistance autoregulation functionality of the natural microcirculation, it is necessary to establish relationships between process parameters and the formation of biomimetic microanatomy (cell organization and phenotype). To gain understanding of how to achieve this functionality in engineered tissue, relationships linking engineering parameters (patterned microchannel architecture, scaffold compliance, SMC seeding process, and post-seeding mechanical stimulation by pulsatile flow) with resultant phenotype, architecture, and contractility of vascular SMCs in a medial layer were established.

Vascular SMCs in vivo are organized along the circumference such that they are transverse to the longitudinal axis of the vessel; this organization is critical to the primary function of these cells, which is to control vessel diameter by exerting forces on local extracellular matrix components or on each other (vasoconstriction or vasodilation). While several approaches to producing such organization have been attempted for millimeter-scale diameter vascular grafts (often relying on rolling up cells aligned on a flat surface or circumferentially oriented nanofibers), to date, there have been no successful efforts demonstrating similar circumferential organization and contractile function for SMCs seeded in microchannels patterned in hydrogels, even as bioprinted microfluidic cell-laden hydrogels continue to become more established as tissue and organ mimics for research and therapeutic applications. Notably, the approaches used for a single straight millimeter-diameter vascular grafts (circumferentially organized topography based on patterning or nanofibers, rolling up thin sheets around a shaft, etc.) do not translate to forming correctly arranged contractile SMC layers in a microvessel network within a hydrogel.

### Background and Significance

Microanatomy of the blood vessel: The vasculature consists of a closed network of branching vessels that support blood flow throughout the body. Vessels of varying sizes and architecture serve different functions; the thin walls of capillaries facilitate exchange of soluble compounds, arterioles regulate vascular resistance by adjusting their lumen diameter, the distensibility of elastic arteries helps dampen pressure pulses from the heart, the muscular arteries distribute blood from the elastic arteries to smaller vessels and actively regulate their diameter to adjust flow, the venules collect deoxygenated blood from the capillaries, and the veins hold the majority of blood volume and some contain valves that prevent backflow. Except for capillaries, which have walls only a single EC thick (and are periodically and discontinuously wrapped by pericytes), the vessels have coaxially organized walls divided into three sections: the tunica intima, tunica media, and tunica adventitia. The intima consists of single-cell thick lining of ECs supported on a thin (˜0.1 μm) basement membrane (internal elastic lamina) composed of several varieties of collagen, fibronectin, elastin and laminin. The medial layer is formed from SMCs, the primary purpose of which is to control vessel tone via vasoconstriction or vasodilation. They are surrounded by a collagenous basement membrane and elastic fibrils which facilitate mechanotransduction as well as the transmission of contractile forces from the cells to the matrix. In larger vessels, particularly muscular arteries, the media contains several layers (25-35) of SMCs, whereas in the smaller arterioles, there are only one or two SMC layers. Compared to muscular arteries, elastic arteries have a larger proportion of collagen and elastin fibers than SMCs, enabling their distensibility. The outermost layer, the tunica adventitia, consists of loose collagen bundles, oriented longitudinally along the vessel, interspersed with fibroblasts and other cells that help modulate vascular SMC activity, facilitate in vascular repair, and produce extracellular matrix components. Each vessel type contains different proportions of each of these three layers, with variation in layer composition and mechanical properties to optimize specific function, but (excluding capillaries) the overall tri-layer architecture is consistent.

### The Critical Roles of Microvasculature: Soluble Compound Exchange and Regulation of Vascular Resistance

The microcirculation serves many purposes. Due to limits imposed by diffusion, cells must generally reside within ˜200 μm of a blood vessel to ensure sufficient exchange of oxygen, nutrients, and waste products. In vivo, capillaries are the site of soluble compound exchange between blood and surrounding tissue, using their small size (and thus high surface area) and high density (several hundred per mm²), as well as their single-EC-thick selectively permeable vessel wall. Similarly, any biomaterial of significant thickness (greater than a few hundred microns) that contains metabolizing, functional cells must also include an artificial microvasculature that allows, via perfusion, delivery of necessary soluble compounds to all cells throughout the scaffold volume; significant efforts have yielded a variety of approaches to manufacture these microvessel networks, coated with ECs to mimic the natural endothelium.

A second but equally important role of the microcirculation is regulation of vascular resistance. The largest pressure drop in the vasculature occurs at the arterioles, and it is here that vascular resistance is regulated. While the ECs serve to modulate the permeability of a lumen wall to various molecules and the adventitial fibroblasts produce and remodel extracellular matrix (and both can regulate SMC behavior), the vascular SMCs themselves exert forces to modulate the lumen size and thus resistance to flow. SMCs exhibit diverse phenotypes on a spectrum ranging from synthetic to contractile Moreover, they retain significant plasticity and are able to reversibly change phenotype in response to a wide range of stimuli. Contractile SMCs are spindle shaped, are not particularly proliferative or migratory, and exhibit high levels of α-smooth muscle actin (α-SMA), smooth muscle-myosin heavy chain (SM-MHC), and other contractile marker proteins. Synthetic SMCs do not contract, but instead secrete ECM components, proliferate, and migrate. They exhibit a cobblestone (or rhomboid) morphology and exhibit a lower level of contractile phenotype marker proteins. Several reviews discuss the genetic basis for and environmental modulation of SMC phenotype.

The smooth muscle layer cooperates with the endothelial layer to regulate both vascular tone and permeability. Many circulating signals for regulation of vascular tone do not work, or do not work correctly, without both layers. For instance, acetylcholine causes vasodilation of intact vessels, but causes constriction of vessels in which the endothelium has been stripped. This cooperation requires both an intact endothelial layer, and a correctly structured smooth muscle layer, with the cells aligned in such a way that they are capable of both signaling correctly to the endothelium as well as generating force.

### Current Approaches for Engineering Microvasculature

Engineered vascular systems not only enable the production of thick cell-laden biomaterials, but additionally allow investigation into the effects of pressure and flow on cell biology; such studies are simply not possible with static culture platforms, and flow in 2D culture systems do not correctly simulate pulsatile pressure, flow and stretch. An endothelialized hydrogel channel (i.e. 3D) has recently been demonstrated that mimics the mechanics of the pulmonary vasculature, and used this system to study the effects of flow and pressure on endothelial dysfunction related to pulmonary arterial hypertension. A similar architecture has been employed to show that iPSC-derived brain microvascular ECs are able to form a robust and long-term barrier in a blood-brain barrier model, and that this requires flow as well as appropriate EC variety; the barrier formed by HUVECs under identical conditions was significantly worse and short-lived. Others have used engineered vascular channels to study thrombosis on a chip, angiogenic sprouting, and other cell response to varying flow. In all of these examples, the intima is mimicked by forming a confluent lining of ECs on the inner wall of the channels. A functional medial layer with biomimetic microanatomy, however, has not been produced.

Efforts to engineer artificial vascular structures can be grouped into two categories: those focusing on macrocirculatory vessels and those focused on the microcirculation. This categorization is logical as the geometry, composition, and function of these two categories of vessels are all quite different (which is why most review articles on the subject delineate between the two). Techniques to engineer straight tubular vessels with lumen diameters at the millimeter or larger scale have been refined over many decades, and thus several approaches to pattern and utilize such structures are already well developed. There are many excellent review articles summarizing these techniques (pouring into a cylindrical mold with a removable centered shaft, rolling a cell-laden sheet or electrospun mat into a cylinder, decellularizing and re-seeding harvested vessels, etc.) As these techniques are quite mature, many researchers have formed vessels with lumen diameter >1 mm containing functional cells. It is emphasized, however, that studies employing such tissue constructs (often called tissue engineered large [>6 mm] or small [<6 mm] vessel grafts) must be distinguished from those employing sub-mm lumen vessels to study microcirculatory phenomena.

Efforts devoted to microvasculature engineering have begun much more recently, with the first publication on a microfluidic biomaterial occurring in 2005. Since then, the field has expanded, with multiple top-down approaches emerging (e.g. patterning via soft lithography, “bioprinting” of cell-laden gels and void spaces, 3D printing, or microfiber spinning of sacrificial templates, optical patterning approaches that can selectively crosslink or photodegrade specialized photosensitive gels), detailed descriptions of these approaches are provided below. While it may be premature to claim that the engineering of a functional endothelium in networks of sub-mm channels in a cell-laden biomaterial is a completely solved problem, it is clear that the significant effort devoted to this task over the past 15 years has rendered formation of an endothelial lining in engineered microvasculature within a hydrogel more routine.

The approaches used to form microvasculature in engineered tissue can generally be grouped into one of two categories: “bottom-up” and “top-down”. Bottom-up approaches take advantage of the natural ability of ECs to form lumen networks via angiogenic sprouting and/or vasculogenesis. Because these processes already occur naturally, the resulting lumen architecture is believed to mimic that of natural capillary beds. Many studies have demonstrated formation of capillary beds in vitro via self-assembly of ECs cultured within a thin hydrogel. These bottom-up methods have the benefit of producing channel architectures dictated by the cells themselves and are elegant in their simplicity, but, to date, they been limited to forming vasculature in thin slabs of hydrogel where diffusion limitations are unimportant in the thickness direction. While such thin cell-laden gels are useful for research purposes, their clinical utility is limited, as is their ability to incorporate heterogeneity or complexity in the thickness direction. Moreover, this approach does not enable multilayer coaxial architecture in the vessel walls, as only capillary-like structures (both in terms of size and microanatomy) are formed.

As an alternative to the bottom-up techniques described above, several researchers have pursued top-down approaches that utilize microfabrication or bioprinting techniques to form microchannels in hydrogels. In these approaches, cells may be mixed in the hydrogel prior to casting or introduced via flow into the microchannels after they are fabricated. Thus, the microchannel architecture is dictated not by self-assembly of the cells, but by the fabrication process. These microfluidic hydrogel approaches can provide immediate convective transport of soluble compounds through the microfluidic channel network, and can thus support all embedded cells instantly. Early work using traditional microfabrication techniques demonstrated the production of 2D microfluidic patterns within cell-laden hydrogels. More recently, approaches employing 3D printing of sacrificial structures have illustrated the importance of picking an appropriate sacrificial material that is both patternable and compatible with cell-laden hydrogels. Bioprinted thermoresponsive polymers have been used as sacrificial templates, as no protective barrier is required and their disintegration may be triggered simply by cooling, an environmental change sufficiently benign such that cell viability is not significantly affected. The channels formed with this approach were shown to enhance cell viability.

Significant success has been achieved in forming channels in cell-laden gels using other thermoresponsive polymers based on Pluronic F127, poly n-isopropylacrylamide (PNIPAM), or polyvinyl caprolactam (PVCL). Additionally, techniques have recently been developed to print sacrificial thermoresponsive polymer templates using fused deposition modelling (FDM), the most widespread additive manufacturing process (e.g. Makerbot, Stratasys, etc.).

Another approach to patterning vasculature utilizes light to either selectively photopolymerize, photodegrade, or ablate material to form channel networks. Early work by Khademhosseini's group demonstrated a stereolithographic approach to patterning GelMA, resulting in hexagonal or woodpile lattices of channels with diameters less than 200 μm. Several studies utilizing photodegradable polymers have demonstrated optical patterning of vascular channels in cell-laden gels. Recent work demonstrated impressive channel network formation using multiphoton lithography and a custom photosensitive gel chemistry. While optical patterning approaches have the potential to achieve resolution sufficient to pattern capillary-sized vessel (and even smaller, if needed), this is generally not demonstrated due to the difficult of seeding cells in such channels.

It is clear that the techniques used to produce microvessel networks are becoming more mature and widespread, attaining higher complexity in network architecture, smaller feature size, and higher throughput. Indeed, many commercial bioprinters (e.g. Inkredible, Allevi, BioBots, EnvisionTEC 3D-Bioplotter, etc.) are capable of working with bioinks specifically devoted to patterning such microvessels (e.g. Pluronic F127 or carbohydrate glass [sacrificial templating], gelatin methacrylate [photopatterning]). In all of the work described above, however, the highest level of microanatomical complexity engineered into the vessel wall itself is simply the formation of an endothelial layer without any additional structure, thereby most closely mimicking capillaries. Except for some previous work employing sacrificial microfibrous templates (which routinely form channel diameters at the 10 μm scale), none of the lumen sizes formed in prior work are appropriate for capillaries; they are too large and instead mimic arterioles and small resistance arteries. This critical mismatch of microvasculature lumen size and vessel wall microanatomy highlights the need to establish techniques enabling production of appropriate microanatomical complexity at the microvessel wall, so as to mimic the coaxial multilayer architecture of natural non-capillary microvessels (resistance vessels) and enable regulation of vessel tone.

Engineering Aligned and/or Coaxially Organized SMCs

The function of most varieties of cells is intimately linked to their morphology and organization within tissue. Thus, many researchers have investigated techniques to pattern these cells such that they align, while maintaining the necessary viability and phenotype to achieve the desired mechanical actuation. One common approach is to pattern “stripes” of adhesion sites or grooves in biomaterial surfaces, resulting in aligned lines of spindle-shaped cells. Similarly, by providing a surface with anisotropic topography, SMCs can be cultured such that they orient along a preferred axis on or in the material. Indeed, the literature describing engineered cell-laden small diameter (mm scale) vascular grafts contains a wide range of approaches, including seeding cells in a gel cast around a freestanding mandrel that is subsequently removed, using electrospun fibers deposited on a rotating mandrel to obtain circumferential alignment that can guide seeded cells, seeding into porous tubes, and seeding one or more layers of cells on a flat sheet that is subsequently rolled around a removable shaft to produce a tube. SMCs have also been seeded onto decellularized arteries (lumen diameters of several mm) in studies demonstrating the importance of pulsatile flow for obtaining a contractile SMC phenotype. These efforts have resulted in some success obtaining circumferentially aligned and, in some cases, coaxially organized layers of cells in larger vessels. These prior approaches, however, do not lend themselves to producing microvessels with coaxial layering and/or circumferential alignment, as the mechanical properties of the materials are insufficient and the manual assembly required incompatible with the delicate nature and small size of microscale vessels. Additionally, it would be impossible to form branching structures, a critical topological feature in microvasculature.

In addition to aligned surfaces, cyclic mechanical stretch can induce alignment of SMCs and has been shown to promote a contractile phenotype, upregulation of genes associated with regulation of tone, and improved cell-cell junctions. Cyclic stretch can be induced either via mechanically pulling on the substrate (typically 2D culture) in a periodic fashion (e.g. Flexcell® platform) or by applying pulsatile flow through a lumen in a 3D matrix with appropriate compliance (an approach that more closely mimics the source of cyclic stretch in vivo). The use of physiological pulsatile flow to achieve cyclic stretch in small vessel (mm-scale lumen) constructs has been shown to enhance formation and function of a medial layer, and promote a contractile SMC phenotype; this mechanism can be used to achieve contractile phenotype in SMCs cultured in arteriole-sized microchannel networks.

There has been some limited previous effort devoted to forming coaxial architecture within a microvessel-like (sub-millimeter) structure formed from a hydrogel. For example, coaxial extrusion approaches are capable of forming free-standing hydrogel tubes that may contain embedded cells. Using a different approach, Khademhosseini and colleagues used GelMA patterned around needles to form a single straight hydrogel channel containing HUVECs in the intima, SMCs in the media, and fibroblasts in the adventitia. While the researchers demonstrated cells viability and an endothelial barrier, no helical SMC organization or function was demonstrated. In another recent example, researchers seeded vascular SMCs on the walls of a microfluidic channel in the elastomer polydimethylsiloxane (the most popular material for forming microfluidics); they found that wrinkles induced in the PDMS channel walls successfully facilitated circumferential organization of seeded SMCs, as well as a contractile phenotype (as indicated by spindle-shaped cells). However, due to the use of stiff PDMS (which, unlike a hydrogel, does not support cells within the matrix or relevant diffusion of soluble compounds), contractile function was not demonstrated. Two recent publications demonstrating coaxial EC/SMC organization via bioprinting also showed their models exhibited a sub-physiological level of contractility; however, there was no data indicating appropriate SMC circumferential alignment or layering (SMCs were embedded in 3D in the bioink hydrogel with vessel wall thickness over 10× greater than natural vessels of similar lumen diameter, a non-physiological architecture) and the influences of matrix properties and flow pulsatility/magnitude were not examined. Another recent publication described the formation of microscale engineered pulmonary artery-like “rolls” (with SMC/EC co-culture) via spontaneous folding of strain-engineered substrates combined with lithographic patterning of fibronectin. While this work achieved impressive similarity to natural vessel cytoarchitecture, it is not clear how individual freestanding rolls could be integrated into a perfusable microvasculature (single vessel or branching), limiting the broader utility of this approach. Another recent study employed both iPSC-derived ECs and SMCs in a microfluidic fibrin hydrogel vasculogenesis model, but the SMCs were dispersed uniformly throughout the hydrogel and did not wrap the microvessels, preventing them from contracting or dilating vessels. Overall, none of the prior literature describing coaxial arrangement of various cell types in an engineered perfusable microvessel has recapitulated the alignment, phenotype, and contractile function of vascular SMCs in the medial layer of resistance vessels in natural tissue.

### Achieving Functional Vascular Smooth Muscle Cells in a Medial Layer

Coaxing SMCs to form an appropriate architecture mimicking the in vivo medial layer within a microchannel in a hydrogel remains a significant challenge. While a great deal of effort has been devoted to seeding larger vascular grafts with SMCs, efforts towards achieving an appropriate medial layer for small resistance artery and arteriole-sized channels within engineered tissue constructs are few and far between, and the results to date have generally shown only that cells can be placed in these channels, not that they are correctly organized or capable of vasoconstriction. Given the importance of the medial layer in the function of non-capillary microvessels (in particular, small resistance arterioles), it is critical to be able to reproduce this microanatomical feature, with correct architecture and function, to engineer biomimetic microvasculature capable of replicating the vessel tone regulation of natural small resistance arteries and arterioles.

### Experimental Approach: Employing ECM Coatings to Achieve Helically Aligned SMCs

The primary hydrogel employed is gelatin, crosslinked enzymatically with microbial transglutaminase (mTG). There is a large base of literature providing compelling data that suggest its tunable stiffness (over 0.1-50 kPa, by adjusting the gelatin and crosslinker concentrations), ability to support contractile SMC phenotype, and general utility for long term 3D cell culture (supporting EC, SMC, and other cell types). It is noted, however, that the present approach to induce SMC helical alignment and contractile phenotype is hydrogel independent, in that it does not depend on the particular matrix composition and can be performed with any hydrogel of appropriate compliance that supports SMC adhesion and viability. As such, several alternate hydrogel compositions (each of which have stiffness tunable independent of density) are envisioned, such as glycated collagen, gelatin methacrylate (compatibility with SMCs already demonstrated), and an interpenetrating network incorporating gelatin and a low concentration of alginate (to achieve high stiffness but maintain accessible RGD sites).

To further adjust mechanical properties, elastin (5%, 15%, 50%, all w/w elastin/gelatin, corresponding to the elastin content of skin, lung, and arterial tissue, respectively) can be added to the gelatin mixture; previous research has suggested that this approach may promote enhanced contractile phenotype.

### Effect of Substrate Stiffness and ECM Coating

In vivo, the phenotype and associated behavior of SMCs in the vascular wall are regulated by a complex interplay of genetic programming with topographical and biochemical cues; While it is clear that extracellular matrix (ECM) composition and organization contribute to phenotypic modulation, there is significant ambiguity on the effects of specific ECM components, with many studies reporting what appears to be either contradictory results or results extremely specific to unique culture conditions. Laminin has been shown to promote both contractile and synthetic phenotypes in different culture systems. Collagen I promotes the synthetic phenotype when in monomeric form but contractile phenotype when in a polymerized form, while collagen type IV has been shown to promote the contractile phenotype. Similarly, matrix compliance seems to have a significant effect on SMC phenotype, but different studies report vastly different moduli for optimum substrate stiffness to promote SMC contractile phenotype, with the effects of matrix stiffness being at least partly dependent on cell density and potentially other factors.

Media used: Several soluble signaling factors play a role in SMC phenotype regulation; platelet-derived growth factor (PDGF), basic fibroblast growth factor (bFGF), insulin-like growth factor (IGF), epidermal growth factor (EGF) have all been implicated in maintenance of the synthetic phenotype. Heparin and transforming growth factor beta 1 (TGF-β) are known to promote contractile phenotype. Commercial media formulations exist for promoting contractile phenotype (smooth muscle differentiation medium [SMDM]-1% v/v Fetal Bovine Serum [FBS] and 30 μg/mL heparin, with no other added growth factors) or maintaining synthetic phenotype (smooth muscle growth medium [SMGM]-5% v/v FBS spiked with bFGF, EGF, IGF, and 5 ng/ml heparin) in SMCs cultured in TCPS flasks (2D, rigid surface, non-physiological).

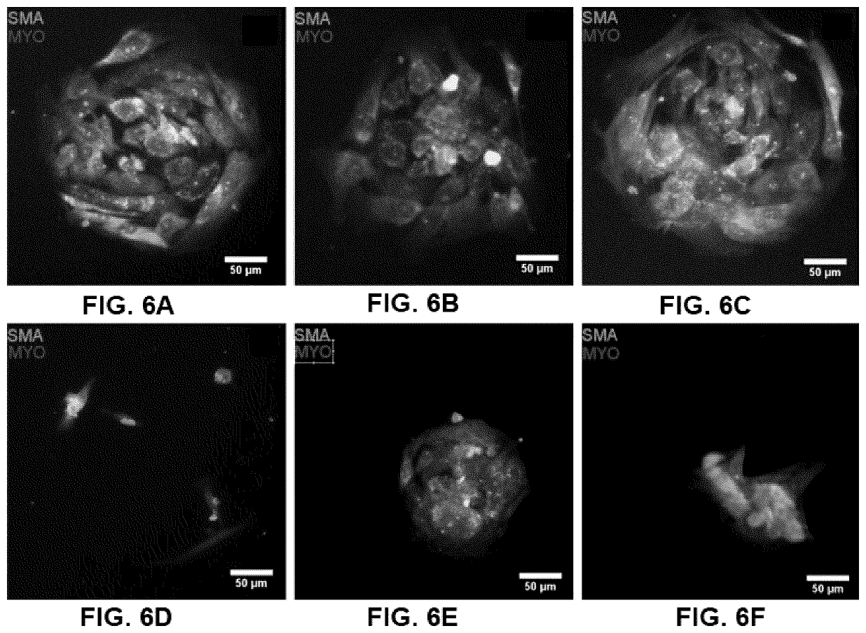

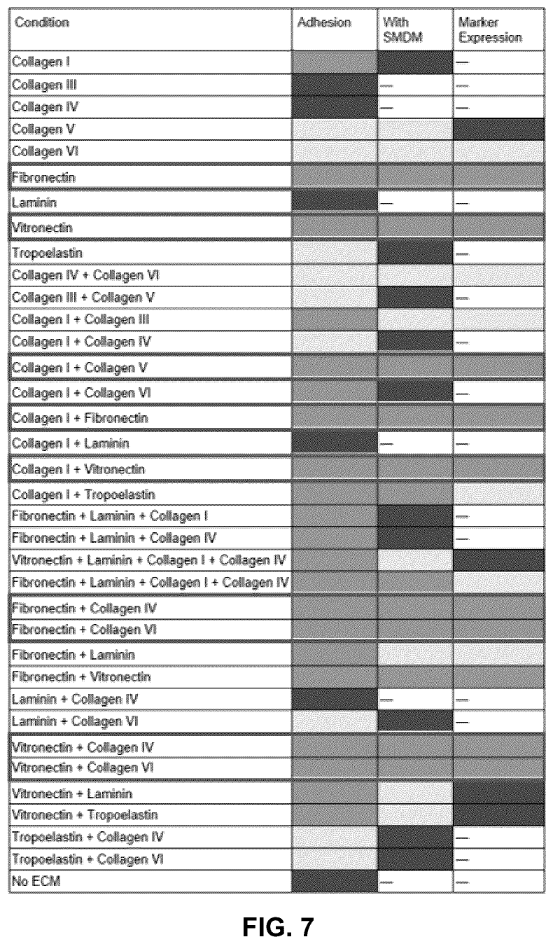

Human Arterial Smooth Muscle Cells (HASMCs) were cultured in flasks with SMGM until the desired quantity of cells was available. HASMCs were then seeded (2.5×10^{5lls/mL) onto a slide coated with} 36 different ECM conditions (ECM Select®) Array Kit Ultra-36, Advanced BioMatrix), including human collagen I, III, IV, V, VI, laminin, fibronectin, vitronectin, and tropoelastin. The cells were then cultured for 2 days in Smooth Muscle Growth Medium [SMGM] and 12 days in Smooth Muscle Differentiation Medium [SMDM] per published protocols; and subsequently stained for contractile markers alpha smooth muscle actin (αSMA) and smooth muscle myosin heavy chain (SM-MHC) (FIGS. 6A-6F contain representative images). Each condition was then graded for initial cell adhesion, cell morphology upon switching to SMDM, and D14 levels of αSMA and SM-MHC (FIG. 7).

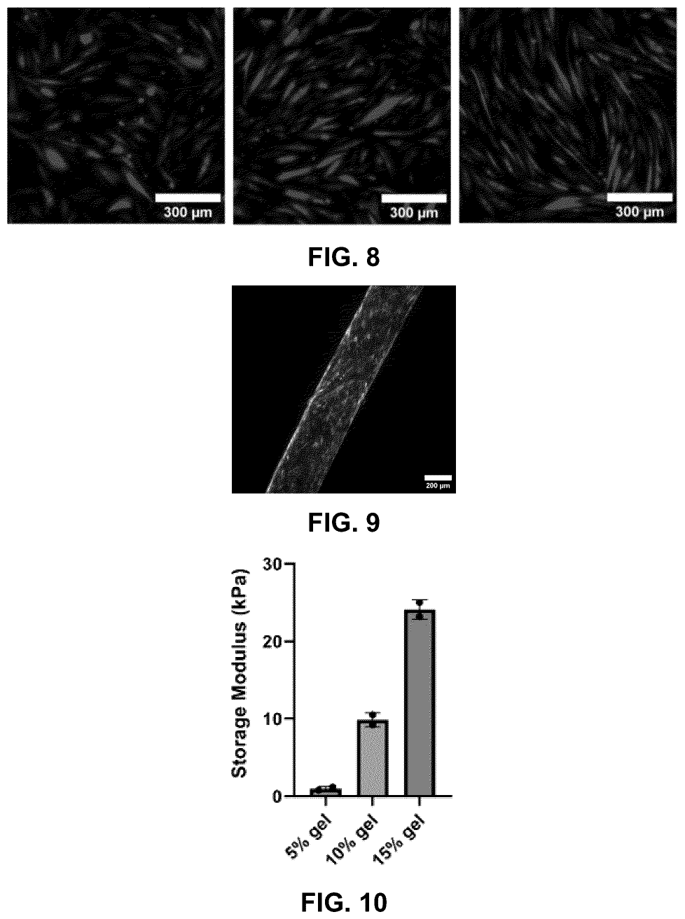

Substrate stiffness screening: While there is consensus that matrix stiffness is a key factor regulating SMC behavior, there is not agreement in the literature on optimal stiffness to promote contractile phenotype in vascular SMCs. To determine the effects of stiffness, PDMS culture plates with storage moduli ranging from 0.2 kPa-64 kPa (CytoSoft® Discovery Kit, Advanced Biomatrix) were used. Given preliminary data regarding ECM composition, plates were coated with collagen IV+fibronectin+vitronectin, after which SMCs were seeded (2.5×10⁵ cells/mL) and cultured (2 days SMGM, 12 days SMDM). Significant differences in cell morphology were observed (FIG. 8).

Hydrogel stiffness tuning: To vary the hydrogel matrix mechanical properties, one gelatin concentration was be chosen (e.g. 10% w/v) and, informed by extensive literature employing mTG-gelatin, the amount of crosslinking enzyme varied (0.1%-2%) to achieve the desired mechanical properties; in this way the crosslink density can be varied without changing the gel concentration (i.e. gel density). If increased stiffness is required, an interpenetrating network incorporating gelatin and a low concentration of alginate can be used. To further adjust mechanical properties, adding elastin (5%, 15%, 50%, all w/w elastin/gelatin, corresponding to the elastin content of skin, lung, and arterial tissue, respectively) to the gelatin mixture can also be investigated; previous research has suggested that this approach may promote contractile phenotype. To verify gel stiffness, unconfined compression tests can be conducted using an Instron 5944. Additionally, shear storage modulus can be measured using a TA Instruments AR-G2 rheometer. By adjusting concentration and crosslinking, matrix stiffness can be tuned such that channel distensibility (as measured by applying 0-80 mmHg pressure and measuring diameter change via microscopy) matches that of natural resistance vessels (0.2-2% per mmHg).

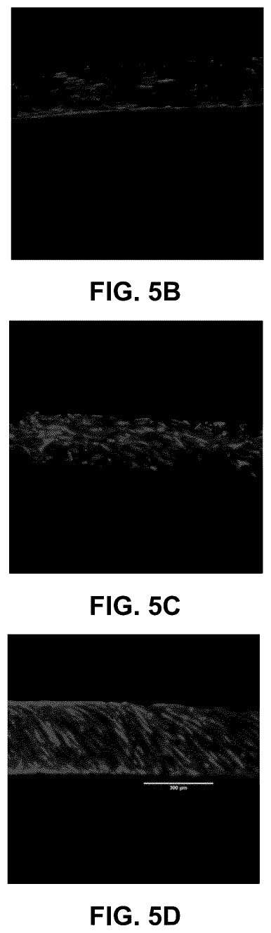

Culture of HASMCs on microchannel walls: Channels were formed in 10% gelatin (crosslinked with mTG), the walls coated with the best performing ECM component combinations, and then HASMCs were seeded on the channel walls. For experiments incorporating a coating of collagen IV, fibronectin, and vitronectin, dramatic helical orientation was observed; this was not observed with the other ECM coatings (FIGS. 5A-5D). However, it has yet to be determined whether the coating composition is what matters, or the total amount of coating. This is the first demonstration of helical SMC architecture in an engineered microvessel without employing patterned topography or adhesion sites. Additionally, helically aligned HASMCs (after 6 days monoculture in SMDM) were coated with a solution of 400 μg/mL collagen IV (incubated 1 hour), after which Human Umbilical Vein Endothelial Cells (HUVECs) were seeded (also introduced by flow). Imaging after 2 days of co-culture indicated robust HUVEC adhesion and spreading similar to what is typically observed in HUVEC monoculture in microchannels (FIG. 9). Details of the patterning and SMC and EC culture approach follow below.

### Channel Device Fabrication (3D)

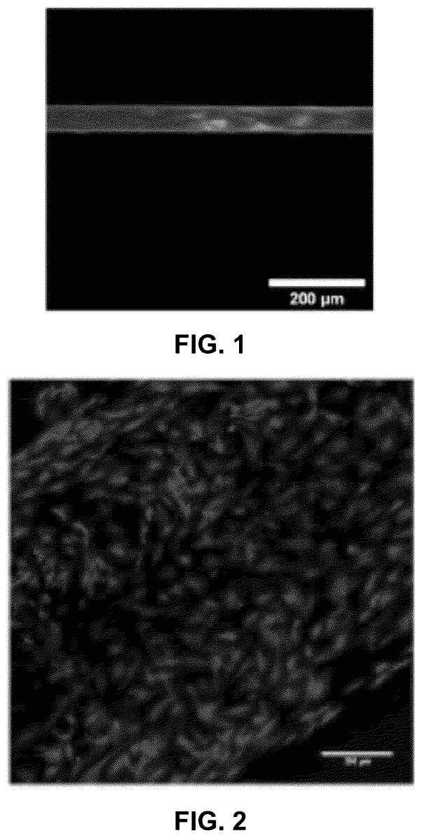

It is straightforward to pattern a single straight microchannel using either a printed sacrificial (dissolvable) template or a removable placeholder. Using a wire, needle, or glass capillary as a removable template, simple straight channels (lumen diameters 50 μm, 100 μm, 200 μm, and 300 μm) can be formed within gelatin-mTG (FIG. 1). Other patterning approaches may be used to make single channels or branching channel networks.

### SMC Monoculture

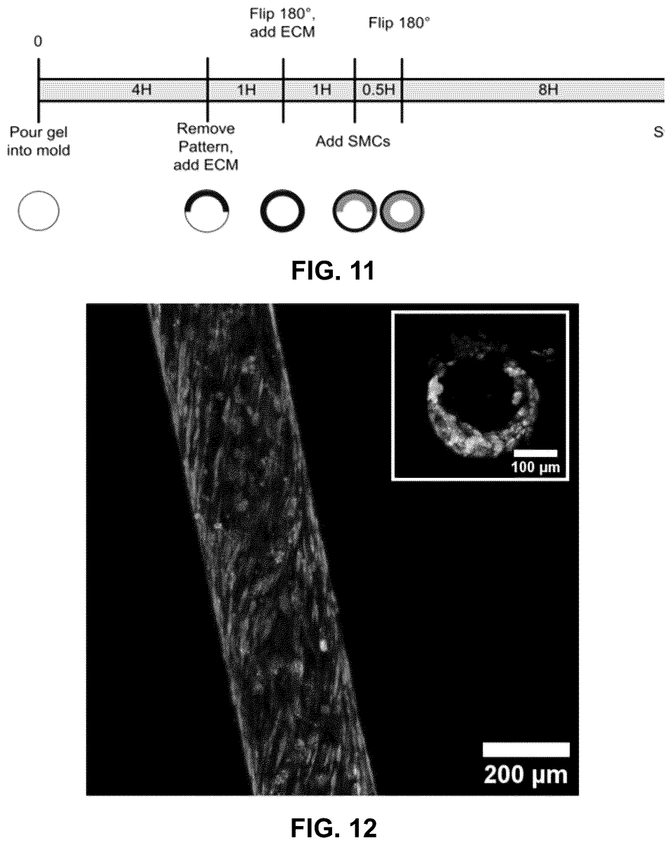

After channels are formed and coated with the desired ECM components, SMCs are introduced into the channel by flow (suspended in SMGM at a density of 5×10⁴ cells/cm² of channel surface area) and allowed to adhere for one hour. The device is flipped 180° and cells will be introduced a second time and again allowed to adhere; based on previous experience with channels of this size range, this approach ensures complete coverage of the channel walls (instead of just the bottom of the channel). Eight hours post-seeding, SMGM is replaced by SMDM. Cells are cultured for up to 14 days. FIG. 11 illustrates this process.

### EC/SMC Co-Culture

Intimal ECs help maintain SMC contractile phenotype both in vitro and in animal models, and play a crucial role in regulating vascular resistance by modulating SMC behavior in response to a wide range of stimuli. As such, it is critical to include them in engineered microvasculature models aiming to faithfully recapitulate natural signaling. Starting with a single channel with a layer of helically oriented SMCs, the inner layer can be coated with a solution of either collagen IV alone or collagen IV+elastin to serve as an elastic lamina, and then HUVECs can be seeded (10⁶ cells/mL allowed to adhere for 1 hour) to form a confluent monolayer.

### Example 2: Relationships Between Dynamic Mechanical Cues and SMC Phenotype and Architecture in a Microfluidic Channel Network within a Hydrogel

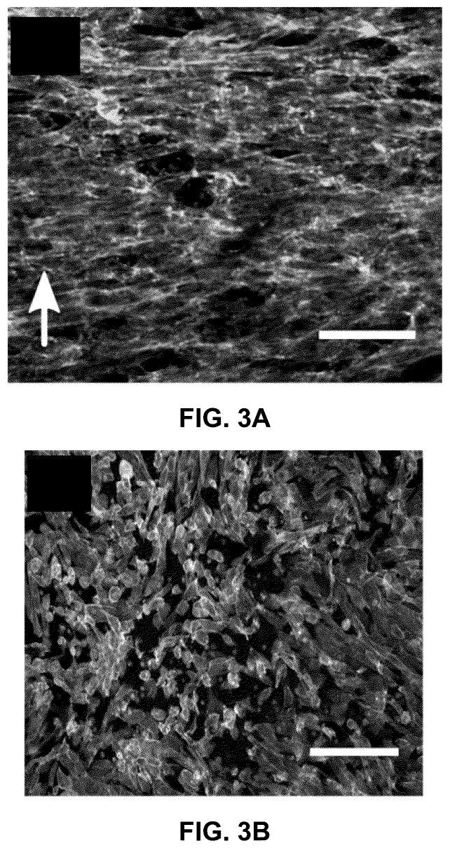

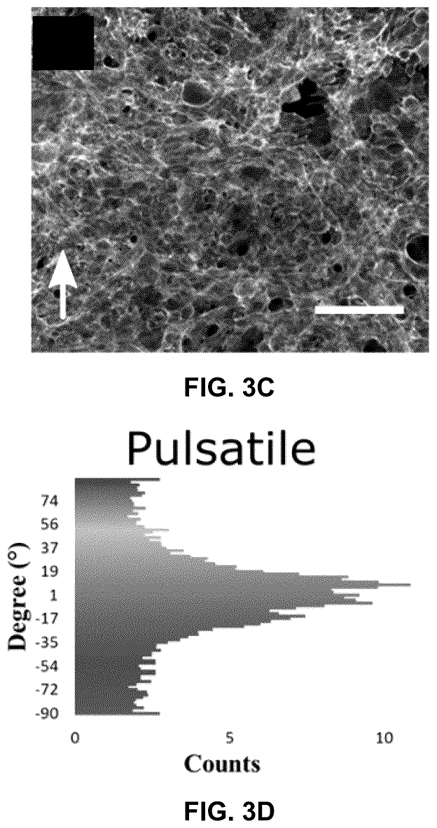

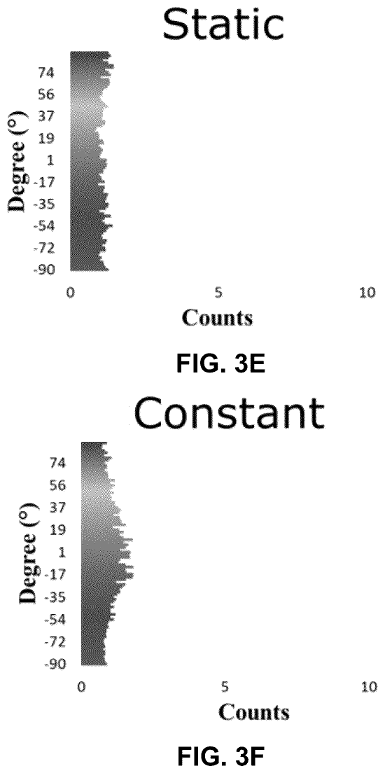

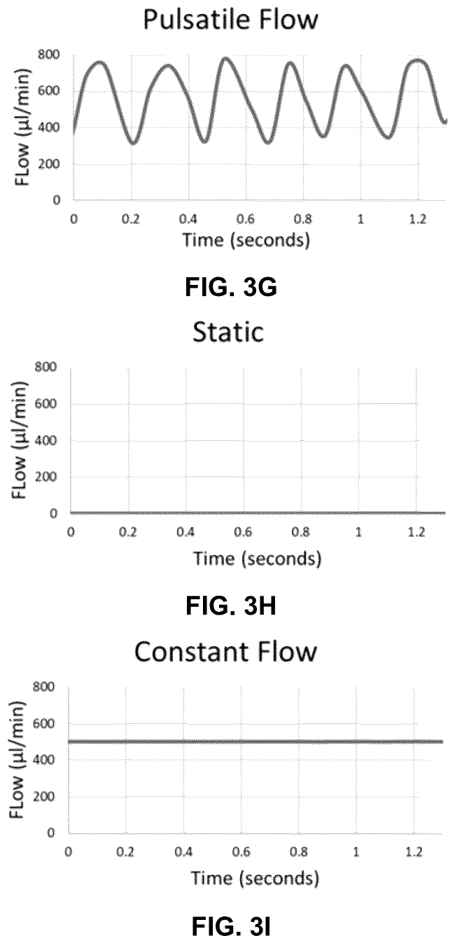

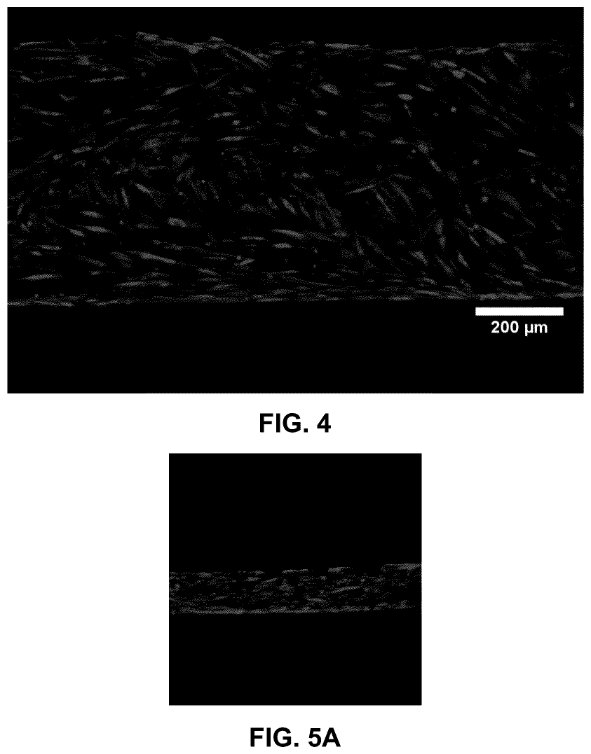

It is well established that the architecture and phenotype of vascular SMCs is, to a meaningful extent, influenced by cyclic stretch caused by pulsatile flow, and several researchers have used this knowledge to facilitate SMC organization in vitro either in 2D or for macrocirculatory vessel geometries. Additionally, in related work, it has been found that pulsatile flow will enhance growth and induce transverse alignment of human ductus arteriosus SMCs cultured in a 3D hydrogel tube (FIGS. 3A-3I) and cause notable changes in expression of ZO-2 (indicative of cell-to-cell contact) and αSMA (indicative of contractile phenotype).

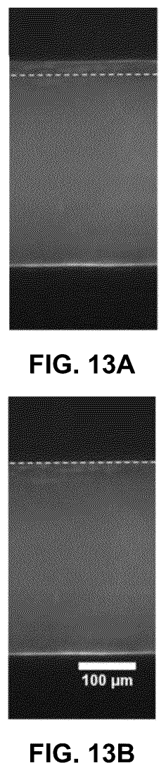

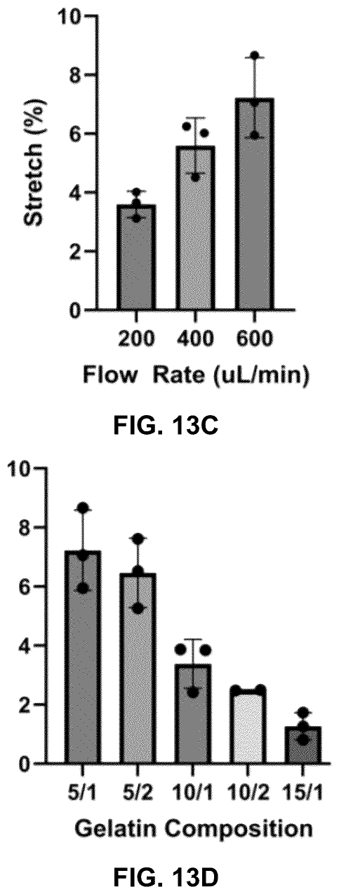

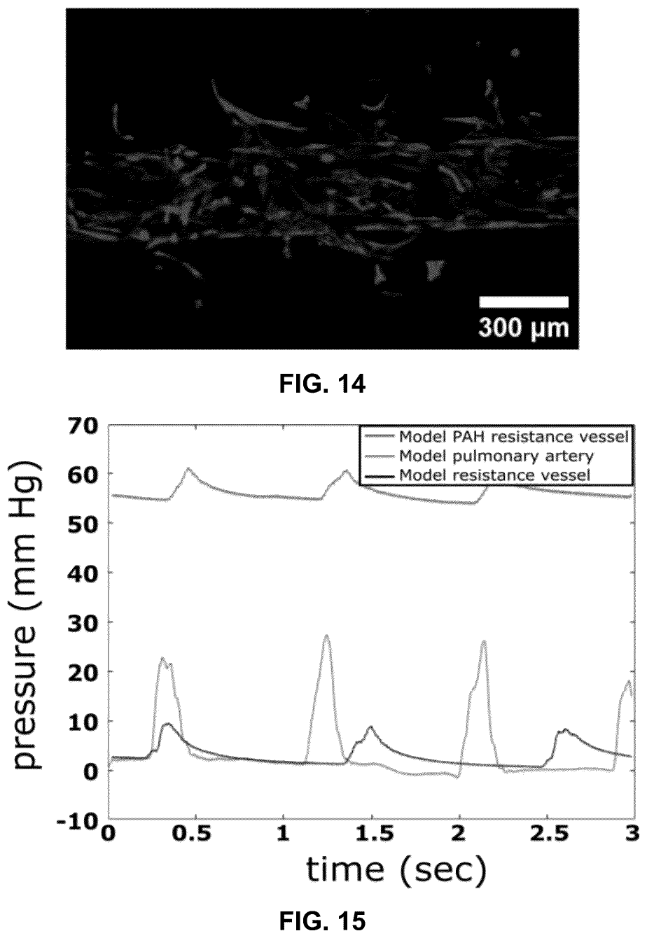

Pulsatile flow containing 2 μm fluorescent polymer beads (Fluospheres) was applied to a 330 μm microchannel formed in mTG-gelatin (of various formulations), and the resulting video data analyzed using the PIVlab suite in Matlab to quantify resulting stretch at the channel wall (FIGS. 13A-13D). This data will allow tuning of matrix properties and flow conditions to achieve the desired wall stretch magnitude and frequency. It has also been found that excessive flow (pathophysiological) drives HASMCs seeded on a microchannel wall to invade the surrounding hydrogel (FIG. 14).

### To Understand and Exploit the Effects of Stiffness, Pulsatile Flow and Cyclic Stretch (Mechanical Cues):

Inspired by these results, the effects of flow, both constant and pulsatile, on the architecture and phenotype of vascular SMCs on the microchannel walls can be leveraged. Flow velocities ranging from 0 (static) to 1 cm/sec may be employed, and pulsatile flow frequency should be roughly 1 Hz (0.5-2 is a reasonable range). In a matrix of suitable distensibility (compliance), this pulsatile flow will produce cyclic strain at the channel walls. In these studies, ˜5% (physiological) strain may be employed, though the effects of 10%, 15%, and 20% strain magnitudes can also be investigated, and strain at 0%, 1%, and 2% can serve as low strain controls. To characterize the time-dependent flow profile within the channel, 100× diluted 2 μm fluorescent polymer beads (Fluospheres) can be imaged and the resulting video data can be analyzed using the PIVlab suite in Matlab. Channel diameters may be imaged using 0.02 μm beads of a different color in the same solution (these will illuminate the entire channel without significantly diffusing into the gel). To measure cyclic stretch, 2 μm fluorescent beads can be mixed in the gelatin solution prior to casting, and utilize confocal microscopy (and image processing algorithms) to quantify the dynamic changes in bead position (thereby indicating strain). Additionally, COMSOL modelling may be used to predict the cyclic strain and fluid shear stresses due to pulsatile flow. In addition to attaining the desired stretch by pulsatile flow, a mechanical stretching apparatus can be built to induce uniaxial cyclic stretch via external means.

Channel Device Fabrication and Cell Seeding: It is straightforward to pattern a single straight microchannel within a cell-laden hydrogel using either a printed sacrificial (dissolvable) template or a removable placeholder. Using a wire, needle, or glass capillary as a removable template, simple straight channels (lumen diameter >50 μm) can be formed within a hydrogel (FIG. 1) and connected to external pumping apparatus. The straight channel geometry may be defined so as to obtain biomimetic pressure and flow behavior representative of a healthy arteriole (e.g. ˜ 40 mmHg drop, ˜1 cm/sec) as measured using a Sensirion USB-interface in-line flowmeter (range 0-1000 μL/min) and a Millar catheter. Branching channel geometries may also be employed, though the downstream effects of pulsatile flow at the inlet will be more complex. Branching channel networks can be formed in hydrogels using a CellInk Bio X6 bioprinter to print Pluronic F127 sacrificial templates. Channel networks can be designed with biologically-inspired architecture branching according to Murray's law and contain vessels with diameters ranging from 500 μm to 70 μm in at least 3 steps. The channel network can be defined to obtain physiological pressure and flow appropriate for a network of small resistance arteries and arterioles (˜60 mmHg between inlet and outlet, ˜3-5 cm/sec at inlet). Additionally, 2 μm Fluospheres may be used to map velocity throughout the network and ensure avoidance of disturbed flow patterns

SMCs are initially cultured in flasks under static conditions and maintained in a more proliferative (i.e. synthetic) phenotype until the desired quantity of cells is available. To seed the SMCs on the channel walls, a suspension of 10⁶ cell/mL is flowed in, allowing the cells to attach to the channel walls (FIG. 2). Cell suspension concentration, flow rate, and length of time allowed for adhesion can all be optimized for particular situations. If the cells settle to the bottom of the channel, seeding may be conducted iteratively, flipping the gelatin slab upside-down on the second seeding (FIG. 11). To achieve multiple layers of SMCs, a solution of extracellular matrix proteins (e.g. collagen, fibronectin, vitronectin, etc.) may be introduced into the channels to coat the cells, after which another seeding of SMCs may be performed; this process can be repeated as many times as layers of SMCs are desired. This layer-by-layer approach has successfully been used in the past to build up several appropriately-organized SMC layers in grooves patterned in poly(t-caprolactone-r-L-lactide-r-glycolide) diacrylate and polyurethane films.

Characterization-Imaging: SMCs should be cultured for at least 3 days under various flow conditions, after which the entire device may be fixed. The cells can be stained for αSMA, SM-MHC, Ki67 (proliferation marker), and meta-vinculin (SMC differentiation marker) and imaged using a confocal microscope. Histological sectioning and imaging (with haematoxylin and eosin staining) can also be used to visualize coaxial organization and layering. Images may be processed with ImageJ to quantify cell alignment relative to the channel, cell elongation (contractile SMCs should be spindle-shaped and quite elongated), relative amount of matrix deposition, and contractile marker expression; these can be determined for experimental conditions consisting of a range of flow velocities, pulsatility, channel geometries, and matrix compliance/channel distensibility (as noted above).

Characterization-Gene expression: The hydrogel can be digested with collagenase and the cells recovered and pelleted. RNA can be extracted, and expression of SMC differentiation and contractile phenotype markers (including smoothelin, calpolin, caldesmon SM-MHC, α-SMA) measured via quantitative RT-PCR. RNAseq (bulk or single cell) may also be used to gain deeper information about changes in gene expression.

Effects of Co-Culture with ECs

It is well established that interactions between SMCs and intimal ECs are critical to proper vessel tone regulation and maintenance of endothelium barrier properties. To engineer this additional complexity and biomimicry into the disclosed model, an intimal layer of ECs can be incorporated interior to the medial layer. Starting with a single channel with 1-3 layers of helically oriented SMCs, the inner layer is first coated with matrix proteins (e.g. elastin and collagen) to serve as an elastic lamina, and ECs are seeded to form a confluent monolayer.

Imaging: Cell viability can be verified using standard live dead stains (e.g. calcein AM and propidium iodide). SMCs can be stained and matrix deposition imaged as described above. ECs can be stained for VE-cadherin, ZO-1, F-actin, and Von Willebrand Factor Collagen and elastin production can also be characterized as described above. Imaging can be performed via confocal microscopy. If required for long term cell tracking, CellTracker dyes can be used or the cells can be transduced with commercially available GFP or RFP lentiviral particles.

Additionally, as co-culture with SMCs is known to modulate EC barrier functionality, the permeability of the endothelium can be characterized in the disclosed model. Cell-lined channels (EC alone or EC/SMC layered co-culture) can be perfused for 1 hour with FITC-dextran (5 mg/mL, Mw=500,000) and imaged every 30 seconds via confocal microscopy. The FITC-dextran leakage can be quantified in ImageJ and Matlab using algorithms previously developed for quantifying permeability in blood-brain barrier models. Fluorescence recovery after photobleaching (FRAP) can be used to quantify the FITC-dextran diffusion coefficient in the gel.

The effects of flow on localized nitric oxide (NO) production can be characterized by fluorescence microscopy imaging of the membrane permeable NO indicator 4,5-diaminofluorescein diacetate (DAF-2 DA), an approach widely used with cultured cells and explanted vessels. The indicator may be added to cell culture media (10 UM) perfused at low and high flow velocities (300 and 1000 μm/sec), and it is expected to see increased fluorescence signal at the higher flow rate. This change can be reduced by including an endothelial nitric oxide synthase (eNOS) inhibitor such as NG-monomethyl-l-arginine (L-NMMA) (500 μM). If enhanced sensitivity is needed, 4-Amino-5-Methylamino-2′,7′-difluorofluorescein diacetate (DAF-FM) may be used. Additionally, to quantify NO production, an amperometric microsensor (WPI ISO-NOP3020) can be placed at the vessel using a micromanipulator

Mechanical testing: To determine vessel wall distensibility, vessel diameter can be measured as a function of pressure (no ECs or SMCs, EC only, SMC only, and EC/SMC), and the data fit to established models.

It is expected that appropriately tuned pulsatile flow will promote enhanced helical organization (closer to 90 degrees with respect to the vessel axis) and enhanced contractile phenotype in the seeded SMCs, whereas constant flow or pulsatile flow with inappropriate waveform will either yield cellular organization and contractile phenotype levels no better than that achieved under static conditions, or worse due to exposing cells to pathophysiological conditions (FIG. 14).

### Example 3: Vasoconstriction and Vasodilation

As highlighted in prior work employing a small collagen-based engineered vessel, contractile SMC phenotype does not necessarily lead to functional vasoconstriction. To demonstrate the ability of the SMCs in the engineered medial layer to correctly respond to stimuli by adjusting vessel tone, the varying ECM coatings and perfusion waveforms are employed as described above and vasoactive agents added to the media after at least 3 days. To induce vasoconstriction, endothelin-1 (ET-1, 500 pM-500 nM) or norepinephrine (10 nM-100 μM) may be used, and to induce vasodilation, the Rho kinase inhibitor HA-1077 (Fasudil) (1-100 μM) or sodium nitroprusside (0.01-1 μM) may be used; these vasoactive agents are known to induce their respective SMC behaviors independent of the presence of an endothelium. To validate EC/SMC interaction, acetylcholine (0.1-10 UM) may be used, as it should induce vasodilation only in coculture with an endothelium. For all vasoactive agents used, studies may be conducted both in monoculture (SMCs only) and in co-culture scenarios (SMC/EC), thereby establishing that the engineered system is capable of replicating the cell-cell interactions critical to proper regulation of vessel tone in response to external stimuli. The concentrations of vasoactive agents listed above are taken from previous experiments examining SMCs or explanted microvessels in vitro and are thus an appropriate range to start; if the desired effects are not observed, the media may need to be spiked with even higher concentrations of these agents.

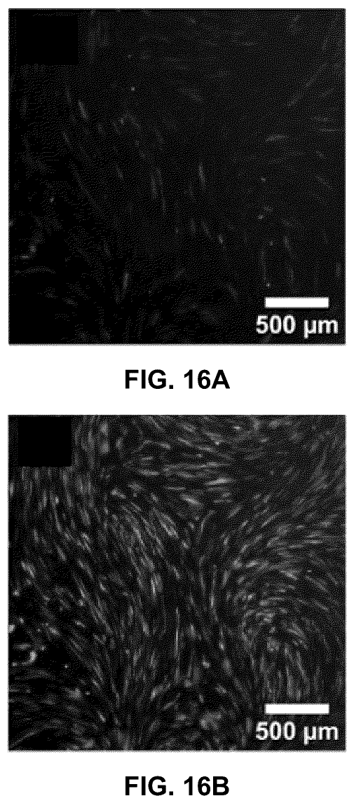

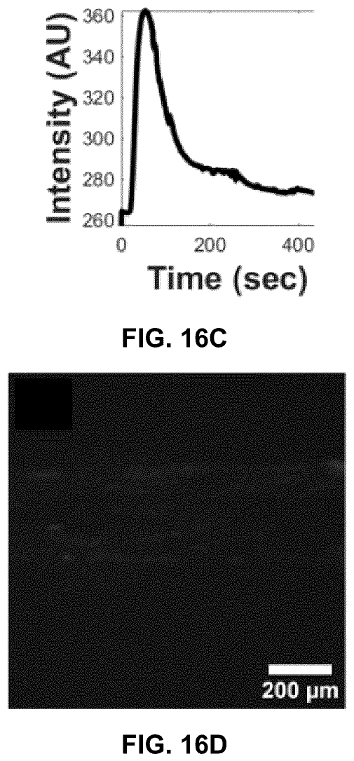

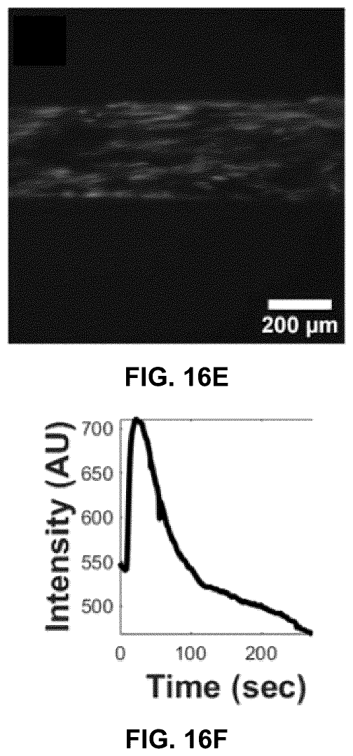

Calcium imaging: HASMCs were infected with lentivirus carrying GCaMP6f and cultured in flasks (2D) and in channels (3D). Cells were then exposed to 500 nM endothelin-1 (ET-1, vasoconstrictor), and imaged via fluorescence microscopy (FIGS. 16A-16F). Quantification of the fluorescence over time indicates substantial intracellular Ca²⁺ release upon ET-1 stimulus.

Characterization of Lumen Diameter: To verify changes in lumen diameter, confocal microscopy is used to image the vessel every 5 minutes after exposure stimulus described above. Image processing algorithms can be used to extract lumen diameter from these images. To validate the resulting change in fluidic resistance, pressure and flow are simultaneously measured using the apparatus described above (Sensirion flow sensor and Millar pressure sensor catheter) (it is expected that changes in the lumen diameter will correlate with measurements of vascular resistance (pressure/flow)). In EC co-culture studies, endothelium permeability after exposure to vasoactive agents can be characterized as described above.

Based on prior studies, it is expected that 10 nM ET-1 should result in ˜17-20% decrease in lumen diameter and that exposure to 10 UM acetylcholine should cause a diameter increase of ˜10-20%; these and similar data can serve as benchmarks for the disclosed engineered vessels.

### Example 4: Engineered Resistance Vessels as Implants for In Vivo Applications

The ultimate goal is to establish, in vivo, the ability of an engineered resistance microvessel to respond correctly to vasoactive stimuli and modulate local blood flow. To demonstrate this, engineered tissue constructs can be implanted in a rodent model, exposed to vasoactive stimuli, and changes in lumen diameter and local blood flow quantified.

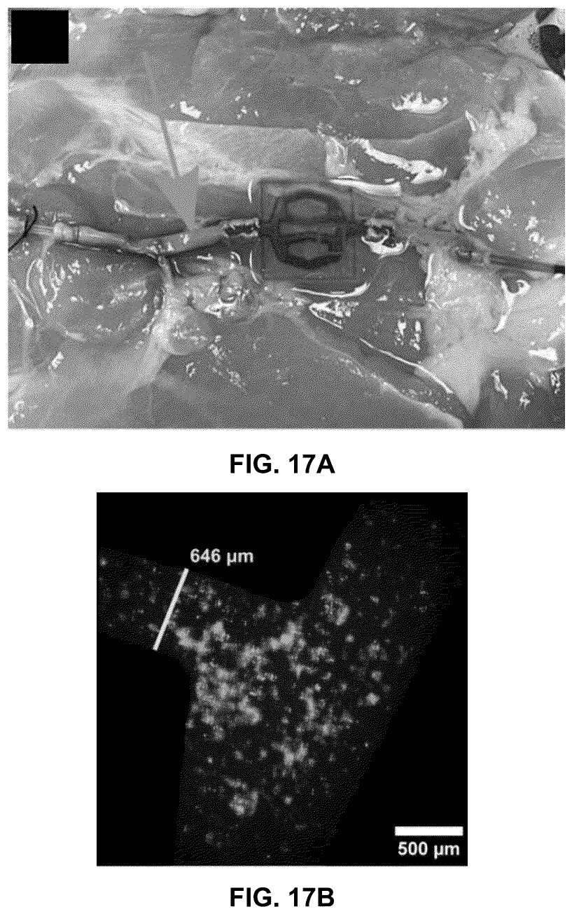

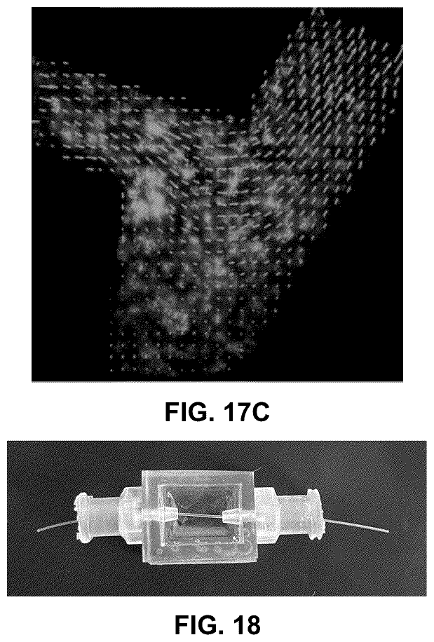

3D printed hydrogel constructs with channel networks embedded within were formed using a modified Phrozen Sonic Mini 8K mSLA 3D printer to photocrosslink polyethylene glycol diacrylate. These constructs were then microsurgically anastomosed to chicken thigh vessels (FIGS. 17A-17C), as this is an established vascular microsurgery training approach. Patency was validated by perfusing with blue dye. Channel diameter and flow were characterized by observing perfused Fluospheres with widefield fluorescence microscopy and analyzing the data using Matlab and ImageJ to track particle movement and vessel size.

Construct Fabrication: Implantable constructs containing either a single straight channel or a branching channel network can be fabricated as per the above, with the inlet and outlet modified to enable surgical anastomosis (FIGS. 17A-17C). Constructs can be seeded with either rat EC/SMC coaxial co-culture or, as a negative control, only rat ECs. Constructs may be cultured in vitro for at least 2 weeks prior to implantation.

Surgical Implantation: Constructs can be microsurgically anastomosed to the femoral artery and vein in the hindlimb of Sprague Dawley rats. Vasoactive Stimuli: 1 hour after implantation, local vasoconstriction can be induced by application of norepinephrine, and local vasodilation can be induced by application of acetylcholine (10 nM-100 UM each). As a positive control, natural small resistance arteries and arterioles branching from the femoral artery in the contralateral hind limb can be exposed to these same vasoactive stimuli, and the response characterized. Vessel Diameter and Blood Flow Measurements: Baseline and changes in both vessel diameter and blood flow can be quantified by imaging injected Fluospheres using widefield fluorescence intravital microscopy. The PIVLab suite in Matlab can be used to quantify Fluosphere velocities.

the constructs with EC/SMC coaxial cocultures mechanically respond to vasoactive stimuli by constriction or dilation of their lumen diameter (resulting in reduced or increased overall blood flow through the construct).

### Example 5: Freestanding Vessels

In the following experiments, porcine skin gelatin, microbial transglutaminase (mTG), and deionized (DI) water were used. Materials and equipment used include rectangular PDMS molds, micropipettors and tips, and serological pipettes.

Gelatin stock was prepared by dissolving gelatin in DI water at the desired concentration, typically 5% (w/v) or higher. The mixture was heated to 55° C. in a water bath and filtered using a 22 μm vacuum filter.

Microbial transglutaminase stock was prepared by dissolving mTG in DI water at 10% (w/v). The mixture was heated to 37° C. in a water bath and filtered using a 22 μm vacuum filter.

The molds used in these experiments were assembled by placing barbs into the holes on opposing ends of rectangular PDMS molds. A piece of nylon monofilament was strung through each barb across the center of the mold (see FIG. 18), leaving excess protruding from each end.

The gelatin stock was poured over the filament, filling the PDMS mold. The gelatin was left at room temperature to allow physical crosslinking to occur. After about 10 min, the filament was gently pulled to one side until the gelatin visibly released. The gelatin was then left at room temperature for another hour and the filament was pulled out, leaving a hollow straight channel behind.

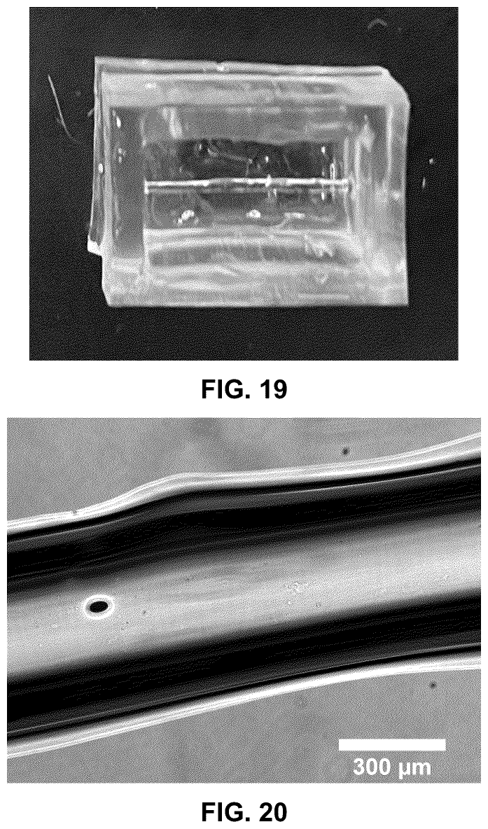

The gelatin tube was formed by using a micropipettor to inject the 10% mTG solution into the channel. The mold was placed into an incubator (5% CO₂, 37° C.). After 5 min, the mTG was flushed out of the channel with DI water. The DI water was then flushed out of the channel by pulsing a bolus of air through using the micropipettor. The mold was placed back into the incubator. Physically crosslinked gelatin melts at this stage, but enzymatic crosslinking occurs where the mTG has penetrated into the gelatin. After 1 hour, the mold is flipped upside down, releasing all non-enzymatically crosslinked gelatin from the mold and leaving a freestanding vessel (see FIGS. 19-20). Elastic moduli of the gels (in kPa) with and without enzymatic crosslinking are presented in FIGS. 21-22.

Fabrication parameters that yield gelatin tubes are presented in Table 1, where an X indicates that a gelatin tube was not successfully formed and a v indicates successful gelatin tube formation.

TABLE 1
Conditions for Enzymatic Gelatin Tube Formation

| | | | | | |
|---|---|---|---|---|---|
| 10 | min | 0.1% mTG | 1% mTG | 5% mTG | 10% mTG |
| 2.5% | gel | X | X | X | X |
| 5% | gel | X | X | √ | √ |
| 10% | gel | X | X | √ | √ |
| 10% | mTG | 2.5% gel | 5% gel | 7.5% gel | 10% gel |
| 1 | min | X | X | √ | √ |
| 5 | min | X | √ | √ | √ |
| 10 | min | X | √ | √ | √ |
| 10% | gel | 30 s | 1 min | 5 min | 10 min |
| 1% | mTG | X | X | X | X |
| 5% | mTG | √ | √ | √ | √ |
| 10% | mTG | √ | √ | √ | √ |

Either before or after tube formation and melting of the surrounding, non-enzymatically-crosslinked medium, the tube can be coated and/or seeded with smooth muscle cells and/or endothelial cells as described in the previous Examples. Additionally, other cell types can be seeded in the hydrogel material by mixing prior to casting the gel over the sacrificial template; such cells will end up embedded in the hydrogel tube (to be distinguished from the smooth muscle cells and/or endothelial cells which are cultured on the interior wall of the tube. Finally, not only can straight tubes be formed by employing straight templates, but branching sacrificial templates can be patterned using known techniques and employed with this approach, resulting in branching networks of tubes.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Amiya, E., et al. The Relationship between Vascular Function and the Autonomic Nervous System. Ann. Vasc. Dis. 7, 109-119 (2014).
2. Angus, J. A., et al. Vasodilatation by acetylcholine is endothelium-dependent: a study by sonomicrometry in canine femoral artery in vivo. J. Physiol. 344, 209-222 (1983).
3. Arakawa, C. K., et al. Multicellular Vascularized Engineered Tissues through User-Programmable Biomaterial Photodegradation. Adv. Mater. 29, 1703156 (2017).
4. Asano, T., et al. A Protein Kinase Inhibitor, Fasudil (AT-877): A Novel Approach to Signal Transduction Therapy. Cardiovasc. Drug Rev. 16, 76-87 (1998).
5. Balikov, D. A., et al. Directing lineage specification of human mesenchymal stem cells by decoupling electrical stimulation and physical patterning on unmodified graphene. Nanoscale 8, 13730-13739 (2016).
6. Balikov, D. A., et al. The nesprin-cytoskeleton interface probed directly on single nuclei is a mechanically rich system. Nucleus 8, 534-547 (2017).
7. Balikov, D. A., et al. Tunable Surface Repellency Maintains Stemness and Redox Capacity of Human Mesenchymal Stem Cells. ACS Appl. Mater. Interfaces 9, 22994-23006 (2017).
8. Beamish, J. A., et al. Molecular Regulation of Contractile Smooth Muscle Cell Phenotype: Implications for Vascular Tissue Engineering. Tissue Eng. Part B Rev. 16, 467-491 (2010).
9. Belo, V. A., et al. Matrix Metalloproteinase 2 as a Potential Mediator of Vascular Smooth Muscle Cell Migration and Chronic Vascular Remodeling in Hypertension. J. Vasc. Res. 52, 221-231 (2015).
10. Bertassoni, L. E., et al. Hydrogel bioprinted microchannel networks for vascularization of tissue engineering constructs. Lab. Chip 14, 2202-2211 (2014).
11. Birukov, K. G., et al. Stretch affects phenotype and proliferation of vascular smooth muscle cells. Mol. Cell. Biochem. 144, 131-139 (1995).
12. Bono, N., et al. A Dual-Mode Bioreactor System for Tissue Engineered Vascular Models. Ann. Biomed. Eng. 45, 1496-1510 (2017).
13. Bosworth, A. M., et al. Modeling Neurovascular Disorders and Therapeutic Outcomes with Human-Induced Pluripotent Stem Cells. Front. Bioeng. Biotechnol. 5, (2018).
14. Bridge, J. C., et al. Electrospun gelatin-based scaffolds as a novel 3D platform to study the function of contractile smooth muscle cells in vitro. Biomed. Phys. Eng. Express 4, 045039 (2018).
15. Broderick, E. P., et al. Enzymatic stabilization of gelatin-based scaffolds. J. Biomed. Mater. Res. 72B, 37-42 (2005).
16. Broillet, M.-C., et al. Photoactivation and calcium sensitivity of the fluorescent NO indicator 4,5-diaminofluorescein (DAF-2): implications for cellular NO imaging. FEBS Lett. 491, 227-232 (2001).
17. Brozovich, F. V., et al. Mechanisms of Vascular Smooth Muscle Contraction and the Basis for Pharmacologic Treatment of Smooth Muscle Disorders. Pharmacol. Rev. 68, 476-532 (2016).
18. Buijtenhuijs, P., et al. Tissue engineering of blood vessels: characterization of smooth-muscle cells for culturing on collagen-and-elastin-based scaffolds. Biotechnol. Appl. Biochem. 39, 141-149 (2004).
19. Cabodi, M., et al. A Microfluidic Biomaterial. J. Am. Chem. Soc. 127, 13788-13789 (2005).
20. Camici, P. G. et al. Coronary Microvascular Dysfunction. N. Engl. J. Med. 356, 830-840 (2007).
21. Catto, V., et al. Vascular Tissue Engineering: Recent Advances in Small Diameter Blood Vessel Regeneration. ISRN Vasc. Med. 2014, 1-27 (2014).
22. Chan, J. M., et al. Engineering of In Vitro 3D Capillary Beds by Self-Directed Angiogenic Sprouting. PLOS ONE 7, e50582 (2012).
23. Chang, W. G. et al. A short discourse on vascular tissue engineering. Npj Regen. Med. 2, 7 (2017).
24. Chan-Park, M. B., et al Biomimetic control of vascular smooth muscle cell morphology and phenotype for functional tissue-engineered small-diameter blood vessels. J. Biomed. Mater. Res. A 88A, 1104-1121 (2009).
25. Chen, P.-Y., et al. Fabrication of large perfusable macroporous cell-laden hydrogel scaffolds using microbial transglutaminase. Acta Biomater. 10, 912-920 (2014).
26. Chen, R., et al. Thermoelectrics of Nanowires. Chem. Rev. (2019). doi: 10.1021/acs.chemrev.8b00627
27. Chłopicki, S. et al. The endothelium-dependent and the endothelium-independent vasodilators in the isolated, perfused guinea pig heart. J. Physiol. Pharmacol. Off. J. Pol. Physiol. Soc. 43, 353-365 (1992).
28. Choi, J. S., et al. Circumferential alignment of vascular smooth muscle cells in a circular microfluidic channel. Biomaterials 35, 63-70 (2014).
29. Chun, Y. W., et al. Differential responses of induced pluripotent stem cell-derived cardiomyocytes to anisotropic strain depends on disease status. J. Biomech. 48, 3890-3896 (2015).
30. Crowder, S. W., et al. Copolymer-Mediated Cell Aggregation Promotes a Proangiogenic Stem Cell Phenotype In Vitro and In Vivo. Adv. Healthc. Mater. 5, 2866-2871 (2016).
31. Cui, H., et al. In vitro and in vivo evaluation of 3D bioprinted small-diameter vasculature with smooth muscle and endothelium. Biofabrication 12, 015004 (2019).
32. D'Amico, R. W., et al. Pulmonary Vascular Platform Models the Effects of Flow and Pressure on Endothelial Dysfunction in BMPR2 Associated Pulmonary Arterial Hypertension. Int. J. Mol. Sci. 19, 2561 (2018).
33. Datta, P., et al. Bioprinting for vascular and vascularized tissue biofabrication. Acta Biomater. 51, 1-20 (2017).
34. Endemann, D. H. et al. Endothelial Dysfunction. J. Am. Soc. Nephrol. 15, 1983-1992 (2004).
35. Faley, S. L., et al. iPSC-Derived Brain Endothelium Exhibits Stable, Long-Term Barrier Function in Perfused Hydrogel Scaffolds. Stem Cell Rep. 12, 474-487 (2019).
36. Feng, J., et al. Quick Layer-by-Layer Assembly of Aligned Multilayers of Vascular Smooth Muscle Cells in Deep Microchannels. Tissue Eng. 13, 1003-1012 (2007).
37. Fisher, S. A. Vascular smooth muscle phenotypic diversity and function. Physiol. Genomics 42A, 169-187 (2010).
38. Furchgott, R. F. et al. The obligatory role of endothelial cells in the relaxation of arterial smooth muscle by acetylcholine. Nature 288, 373-376 (1980).
39. Gao, Y. Biology of Vascular Smooth Muscle: Vasoconstriction and Dilatation. (Springer Singapore, 2017). doi: 10.1007/978-981-10-4810-4
40. Gauvin, R., et al. Microfabrication of complex porous tissue engineering scaffolds using 3D projection stereolithography. Biomaterials 33, 3824-3834 (2012).
41. Ghezzi, C. E., et al. An airway smooth muscle cell niche under physiological pulsatile flow culture using a tubular dense collagen construct. Biomaterials 34, 1954-1966 (2013).
42. Golden, A. P. et al. Fabrication of microfluidic hydrogels using molded gelatin as a sacrificial element. Lab. Chip 7, 720 (2007).
43. González-Avalos, P. et al. Quantification of substrate and cellular strains in stretchable 3D cell cultures: an experimental and computational framework: QUANTIFICATION OF SUBSTRATE AND CELLULAR STRAINS IN STRETCHABLE 3D CELL CULTURES. J. Microsc. 266, 115-125 (2017).
44. Gupta, M. K., et al. Gradient release of cardiac morphogens by photo-responsive polymer micelles for gradient-mediated variation of embryoid body differentiation. J. Mater. Chem. B 5, 5206-5217 (2017).
45. Gupta, M. K., et al. Recent strategies to design vascular theranostic nanoparticles. Nanotheranostics 1, 166-177 (2017).
46. Hall, M. S., et al. Mapping Three-Dimensional Stress and Strain Fields within a Soft Hydrogel Using a Fluorescence Microscope. Biophys. J. 102, 2241-2250 (2012).
47. Hall, M. S., et al. Toward single cell traction microscopy within 3D collagen matrices. Exp. Cell Res. 319, 2396-2408 (2013).
48. Hao H. et al. Arterial Smooth Muscle Cell Heterogeneity. Arterioscler. Thromb. Vasc. Biol. 23, 1510-1520 (2003).
49. Hasan, A., et al. A multilayered microfluidic blood vessel-like structure. Biomed. Microdevices 17, 88 (2015).
50. Hasan, A., et al. Electrospun Scaffolds for Tissue Engineering of Vascular Grafts. Acta Biomater. 10, (2014).
51. Haurani, M. J. et al. Adventitial fibroblast reactive oxygen species as autacrine and paracrine mediators of remodeling: Bellwether for vascular disease? Cardiovasc. Res. 75, 679-689 (2007).
52. Heintz, K. A., et al. Fabrication of 3D Biomimetic Microfluidic Networks in Hydrogels. Adv. Healthc. Mater. 5, 2153-2160 (2016).
53. Hinton, T. J., et al. Three-dimensional printing of complex biological structures by freeform reversible embedding of suspended hydrogels. Sci. Adv. 1, e1500758 (2015).
54. Hippalgaonkar, K., et al. in Therm. Transp. Carbon-Based Nanomater. (ed. Zhang, G.) 319-357 (Elsevier, 2017). doi: 10.1016/B978-O-32-346240-2.00012-1
55. Hsu, Y.-H., et al. A microfluidic platform for generating large-scale nearly identical human microphysiological vascularized tissue arrays. Lab. Chip 13, 2990-2998 (2013).
56. Huang, A., et al. Superoxide Released to High Intra-arteriolar Pressure Reduces Nitric Oxide-Mediated Shear Stress- and Agonist-Induced Dilations. Circ. Res. 83, 960-965 (1998).
57. In Jeong, S., et al. Tissue-engineered vascular grafts composed of marine collagen and PLGA fibers using pulsatile perfusion bioreactors. Biomaterials 28, 1115-1122 (2007).
58. Irvine, S. A., et al. Printing cell-laden gelatin constructs by free-form fabrication and enzymatic protein crosslinking. Biomed. Microdevices 17, 16 (2015).
59. Isenberg B. C. et al. Small-Diameter Artificial Arteries Engineered In Vitro. Circ. Res. 98, 25-35 (2006).
60. Iwasaki K. et al. Bioengineered Three-Layered Robust and Elastic Artery Using Hemodynamically-Equivalent Pulsatile Bioreactor. Circulation 118, S52-S57 (2008).
61. Jain, A., et al. Primary Human Lung Alveolus-on-a-chip Model of Intravascular Thrombosis for Assessment of Therapeutics. Clin. Pharmacol. Ther. 103, 332-340 (2018).
62. Jeong, S. I., et al. Mechano-active tissue engineering of vascular smooth muscle using pulsatile perfusion bioreactors and elastic PLCL scaffolds. Biomaterials 26, 1405-1411 (2005).
63. Kannan, R. Y., et al. The roles of tissue engineering and vascularisation in the development of micro-vascular networks: a review. Biomaterials 26, 1857-1875 (2005).
64. Kim, B.-S., et al. Cyclic mechanical strain regulates the development of engineered smooth muscle tissue. Nat. Biotechnol. 17, 979-983 (1999).
65. Kirby, B. S., et al. Robust Internal Elastic Lamina Fenestration in Skeletal Muscle Arteries. PLOS ONE 8, e54849 (2013).
66. Klabunde, R. Cardiovascular Physiology Concepts. (Lippincott Williams & Wilkins, 2011).
67. Kloxin, A. M., et al. Photodegradable Hydrogels for Dynamic Tuning of Physical and Chemical Properties. Science 324, 59-63 (2009).
68. Koch, T. M., et al. 3D Traction Forces in Cancer Cell Invasion. PLOS ONE 7, e33476 (2012).
69. Kolesky, D. B., et al. 3D Bioprinting of Vascularized, Heterogeneous Cell-Laden Tissue Constructs. Adv. Mater. 26, 3124-3130 (2014).
70. Kolesky, D. B., et al. Three-dimensional bioprinting of thick vascularized tissues. Proc. Natl. Acad. Sci. 201521342 (2016). doi: 10.1073/pnas.1521342113
71. Kurpinski, K., et al. Regulation of Vascular Smooth Muscle Cells and Mesenchymal Stem Cells by Mechanical Strain. 14 (2006).
72. Kurzen, H., et al. Tightening of Endothelial Cell Contacts: A Physiologic Response to Cocultures with Smooth-Muscle-Like 10T1/2 Cells. J. Invest. Dermatol. 119, 143-153 (2002).
73. L′heureux, N., et al. A completely biological tissue-engineered human blood vessel. FASEB J. 12, 47-56 (1998).
74. L′Heureux, N., et al. Human tissue-engineered blood vessels for adult arterial revascularization. Nat. Med. 12, 361-365 (2006).
75. L′Heureux, N., et al. In vitro construction of a human blood vessel from cultured vascular cells: A morphologic study. J. Vasc. Surg. 17, 499-509 (1993).
76. Lee, J. B., et al. Development of 3D Microvascular Networks Within Gelatin Hydrogels Using Thermoresponsive Sacrificial Microfibers. Adv. Healthc. Mater. 5, 781-785 (2016).
77. Lee, J.-J., et al. Dehomogenized Elastic Properties of Heterogeneous Layered Materials in AFM Indentation Experiments. Biophys. J. 114, 2717-2731 (2018).
78. Lee, K. H., et al. Synthesis of Cell-Laden Alginate Hollow Fibers Using Microfluidic Chips and Microvascularized Tissue-Engineering Applications. Small 5, 1264-1268 (2009).
79. Lee, S., et al. Aligned microribbon-like hydrogels for guiding three-dimensional smooth muscle tissue regeneration. J. Biomed. Mater. Res. A 104, 1064-1071 (2016).
80. Lee, Y., et al. Heparin-functionalized polymer graft surface eluting MK2 inhibitory peptide to improve hemocompatibility and anti-neointimal activity. J. Control. Release Off. J. Control. Release Soc. 266, 321-330 (2017).
81. Legant, W. R., et al. Measurement of mechanical tractions exerted by cells in three-dimensional matrices. Nat. Methods 7, 969-971 (2010).
82. Lehr, H.-A., et al. Microcirculatory dysfunction in sepsis: a pathogenetic basis for therapy? J. Pathol. 190, 373-386 (2000).
83. Liao, S. W., et al. Mechanical regulation of matrix reorganization and phenotype of smooth muscle cells and mesenchymal stem cells in 3D matrix. in 26th Annu. Int. Conf. IEEE Eng. Med. Biol. Soc. 2, 5024-5027 (2004).
84. Lilly, B. We Have Contact: Endothelial Cell-Smooth Muscle Cell Interactions. Physiology 29, 234-241 (2014).
85. Liu, W. F. Mechanical regulation of cellular phenotype: implications for vascular tissue regeneration. Cardiovasc. Res. 95, 215-222 (2012).
86. Luo, Y., et al. Direct Plotting of Three-Dimensional Hollow Fiber Scaffolds Based on Concentrated Alginate Pastes for Tissue Engineering. Adv. Healthc. Mater. 2, 777-783 (2013).
87. Ma, J., et al. Thermal conductivity of electrospun polyethylene nanofibers. Nanoscale 7, 16899-16908 (2015).
88. Marelli, B., et al. Compliant electrospun silk fibroin tubes for small vessel bypass grafting. Acta Biomater. 6, 4019-4026 (2010).
89. Martinez-Lemus, L. A. The Dynamic Structure of Arterioles. Basic Clin. Pharmacol. Toxicol. 110, 5-11 (2012).
90. Mason, B. N., et al. Tuning three-dimensional collagen matrix stiffness independently of collagen concentration modulates endothelial cell behavior. Acta Biomater. 9, 4635-4644 (2013).
91. Matsumoto, T., et al. Residual stress and strain in the lamellar unit of the porcine aorta: experiment and analysis. J. Biomech. 37, 807-815 (2004).
92. McDermott, M. K., et al. Mechanical Properties of Biomimetic Tissue Adhesive Based on the Microbial Transglutaminase-Catalyzed Crosslinking of Gelatin. Biomacromolecules 5, 1270-1279 (2004).
93. Miller, J. S., et al. Rapid casting of patterned vascular networks for perfusable engineered three-dimensional tissues. Nat. Mater. 11, 768-774 (2012).
94. Moya, M. L., et al. In Vitro Perfused Human Capillary Networks. Tissue Eng. Part C Methods 19, 730-737 (2013).
95. Mulvany, M. J. et al. Structure and function of small arteries. Physiol. Rev. 70, 921-961 (1990).
96. Murray, C. D. The physiological principle of minimum work: I. The vascular system and the cost of blood volume. Proc. Natl. Acad. Sci. U.S.A. 12, 207 (1926).
97. Murray, C. D. The physiological principle of minimum work: II. Oxygen exchange in capillaries. Proc. Natl. Acad. Sci. U.S.A. 12, 299 (1926).
98. Nagumo, H., et al. Rho kinase inhibitor HA-1077 prevents Rho-mediated myosin phosphatase inhibition in smooth muscle cells. Am. J. Physiol. Cell Physiol. 278, C57-65 (2000).
99. Nichol, J. W., et al. Cell-laden microengineered gelatin methacrylate hydrogels. Biomaterials 31, 5536-5544 (2010).
100. Niklason, L. E., et al. Functional Arteries Grown in Vitro. Science 284, 489-493 (1999).
101. Novosel, E. C., et al. Vascularization is the key challenge in tissue engineering. Adv. Drug Deliv. Rev. 63, 300-311 (2011).
102. O'Grady, B., et al. A Customizable, Low-Cost Perfusion System for Sustaining Tissue Constructs. SLAS Technol. Transl. Life Sci. Innov. 2472630318775059 (2018). doi: 10.1177/2472630318775059
103. O'Rourke, M. F., et al. Clinical applications of arterial stiffness; definitions and reference values. Am. J. Hypertens. 15, 426-444 (2002).
104. Owens, G. K. Regulation of differentiation of vascular smooth muscle cells. Physiol. Rev. 75, 487-517 (1995).
105. Owens, G. K., et al. Molecular Regulation of Vascular Smooth Muscle Cell Differentiation in Development and Disease. Physiol. Rev. 84, 767-801 (2004).
106. Paguirigan, A. et al. Gelatin based microfluidic devices for cell culture. Lab. Chip 6, 407 (2006).
107. Paguirigan, A. L. et al. Protocol for the fabrication of enzymatically crosslinked gelatin microchannels for microfluidic cell culture. Nat Protoc. 2, 1782-1788 (2007).
108. Peyton, S. R., et al. The effects of matrix stiffness and RhoA on the phenotypic plasticity of smooth muscle cells in a 3-D biosynthetic hydrogel system. Biomaterials 29, 2597-2607 (2008).
109. Price, G. M., et al. Effect of mechanical factors on the function of engineered human blood microvessels in microfluidic collagen gels. Biomaterials 31, 6182-6189 (2010).
110. Qi, Y.-X., et al. Cyclic strain modulates migration and proliferation of vascular smooth muscle cells via Rho-GDIα, Rac1, and p38 pathway. J. Cell. Biochem. 109, 906-914 (2010).
111. Qiu, J., et al. Biomechanical regulation of vascular smooth muscle cell functions: from in vitro to in vivo understanding. J. R. Soc. Interface 11, (2014).
112. Rayatpisheh, S., et al. Aligned 3D human aortic smooth muscle tissue via layer by layer technique inside microchannels with novel combination of collagen and oxidized alginate hydrogel. J. Biomed. Mater. Res. A 98A, 235-244 (2011).
113. Rayatpisheh, S., et al. Combining cell sheet technology and electrospun scaffolding for engineered tubular, aligned, and contractile blood vessels. Biomaterials 35, 2713-2719 (2014).
114. Reho, J. J., et al. Smooth muscle contractile diversity in the control of regional circulations. Am. J. Physiol.-Heart Circ. Physiol. 306, H163-H172 (2013).
115. Rensen, S. S. M., et al. Regulation and characteristics of vascular smooth muscle cell phenotypic diversity. Neth. Heart J. 15, 100-108 (2007).
116. Risinger, G. M., et al. Matrix Metalloproteinase-2 Expression by Vascular Smooth Muscle Cells Is Mediated by Both Stimulatory and Inhibitory Signals in Response to Growth Factors. J. Biol. Chem. 281, 25915-25925 (2006).
117. Ryan, A. J. et al. Insoluble elastin reduces collagen scaffold stiffness, improves viscoelastic properties, and induces a contractile phenotype in smooth muscle cells. Biomaterials 73, 296-307 (2015).
118. Rzucidlo, E. M., et al. Regulation of vascular smooth muscle cell differentiation. J. Vasc. Surg. 45, A25-A32 (2007).
119. S. Kinstlinger, et al. 3D-printed fluidic networks as vasculature for engineered tissue. Lab. Chip 16, 2025-2043 (2016).
120. Sasaki, Y. et al. Inhibition of myosin light chain phosphorylation in cultured smooth muscle cells by HA1077, a new type of vasodilator. Biochem. Biophys. Res. Commun. 171, 1182-1187 (1990).
121. Sazonova, O. V., et al. Cell-Cell Interactions Mediate the Response of Vascular Smooth Muscle Cells to Substrate Stiffness. Biophys. J. 101, 622-630 (2011).
122. Schutte, S. C., et al. Tissue engineering of a collagen-based vascular media. Organogenesis 6, 204-211 (2010).
123. Scott, D., et al. High pulsatility flow stimulates smooth muscle cell hypertrophy and contractile protein expression. Am. J. Physiol. Lung Cell. Mol. Physiol. 304, L70-81 (2013).
124. Seliktar, D., et al. Dynamic Mechanical Conditioning of Collagen-Gel Blood Vessel Constructs Induces Remodeling In Vitro. Ann. Biomed. Eng. 28, 351-362 (2000).
125. Sellke, F. W., et al. The Pathophysiology of Vasomotor Dysfunction. Ann. Thorac. Surg. 64, S9-S15 (1997).
126. Sheng, Y. et al. The crosstalk between autonomic nervous system and blood vessels. Int. J. Physiol. Pathophysiolo
127. Sherman, T. F. On connecting large vessels to small. The meaning of Murray's law. J. Gen. Physiol. 78, 431-453 (1981).
128. Shin, S.-J., et al. “On the Fly” Continuous Generation of Alginate Fibers Using a Microfluidic Device. Langmuir 23, 9104-9108 (2007).
129. Shrike Zhang, Y., et al. Bioprinted thrombosis-on-a-chip. Lab. Chip 16, 4097-4105 (2016).
130. Song, J. W. et al. Fluid forces control endothelial sprouting. Proc. Natl. Acad. Sci. 108, 15342-15347 (2011).
131. Spronck, B. et al. Arterial Stiffness: Different Metrics, Different Meanings. J. Biomech. Eng. 141, (2019).
132. Stegemann, J. P. et al. Phenotype Modulation in Vascular Tissue Engineering Using Biochemical and Mechanical Stimulation. Ann. Biomed. Eng. 31, 391-402 (2003).
133. Stegemann, J. P., et al. Mechanical, biochemical, and extracellular matrix effects on vascular smooth muscle cell phenotype. J. Appl. Physiol. 98, 2321-2327 (2005).
134. Stenmark, K. R., et al. The adventitia: essential regulator of vascular wall structure and function. Annu. Rev. Physiol. 75, 23-47 (2013).
135. Sugiura, S., et al. Tubular gel fabrication and cell encapsulation in laminar flow stream formed by microfabricated nozzle array. Lab. Chip 8, 1255-1257 (2008).
136. Tesfamariam B et al. Endothelium-dependent and endothelium-independent vasodilation in resistance arteries from hypertensive rats. Hypertension 11, 440-444 (1988).
137. Thakar, R. G. et al. Cell-Shape Regulation of Smooth Muscle Cell Proliferation. Biophys. J. 96, 3423-3432 (2009).
138. Thielicke, W. et al. PIVlab-Towards User-friendly, Affordable and Accurate Digital Particle Image Velocimetry in MATLAB. J. Open Res. Softw. 2, e30 (2014).
139. Tijore, A., et al. Bioprinted gelatin hydrogel platform promotes smooth muscle cell contractile phenotype maintenance. Biomed. Microdevices 20, 32 (2018).
140. Traore, M. A. et al. Tissue Engineering the Vascular Tree. Tissue Eng. Part B Rev. (2017). doi: 10.1089/ten.teb.2017.0010
141. Truskey, G. A. Endothelial Cell Vascular Smooth Muscle Cell Co-Culture Assay For High Throughput Screening Assays For Discovery of Anti-Angiogenesis Agents and Other Therapeutic Molecules. Int. J. High Throughput Screen. 2010, 171-181 (2010).
142. Uttayarat, P., et al. Micropatterning of three-dimensional electrospun polyurethane vascular grafts. Acta Biomater. 6, 4229-4237 (2010).
143. Vanhoutte, P. M., et al. Modulation of Vascular Smooth Muscle Contraction by the Endothelium. Annu. Rev. Physiol. 48, 307-320 (1986).
144. W. Alford, P., et al. Vascular smooth muscle contractility depends on cell shape. Integr. Biol. 3, 1063-1070 (2011).
145. Wang, G., et al. Origin and differentiation of vascular smooth muscle cells. J. Physiol. 593, 3013-3030 (2015).
146. Wasner, G. Vasomotor Disturbances in Complex Regional Pain Syndrome-A Review. Pain Med. 11, 1267-1273 (2010).
147. Wen, C., et al. Mechanically Robust Gelatin-Alginate IPN Hydrogels by a Combination of Enzymatic and lonic Crosslinking Approaches. Macromol. Mater. Eng. 299, 504-513 (2014).
148. Wollner, L., et al. Failure of cerebral autoregulation as a cause of brain dysfunction in the elderly. Br Med J 1, 1117-1118 (1979).
149. Xu, C. Y., et al. Aligned biodegradable nanofibrous structure: a potential scaffold for blood vessel engineering. Biomaterials 25, 877-886 (2004).
150. Xu, J. et al. Vascular wall extracellular matrix proteins and vascular diseases. Biochim. Biophys. Acta 1842, 2106-2119 (2014).
151. Xu, Z. C., et al. Engineering of an elastic large muscular vessel wall with pulsatile stimulation in bioreactor. Biomaterials 29, 1464-1472 (2008).
152. Yajima, Y., et al. Facile fabrication processes for hydrogel-based microfluidic devices made of natural biopolymers. Biomicrofluidics 8, 024115 (2014).
153. Yang, L., et al. Thermal conductivity of individual silicon nanoribbons. Nanoscale 8, 17895-17901 (2016).
154. Yazdani, S. K., et al Smooth Muscle Cell Seeding of Decellularized Scaffolds: The Importance of Bioreactor Preconditioning to Development of a More Native Architecture for Tissue-Engineered Blood Vessels. Tissue Eng. Part A 15, 827-840 (2009).
155. Ye, G. J. C., et al. The contractile strength of vascular smooth muscle myocytes is shape dependent. Integr. Biol. 6, 152-163 (2014).
156. Yen, W., et al. Endothelial Surface Glycocalyx Can Regulate Flow-Induced Nitric Oxide Production in Microvessels In Vivo. PLOS ONE 10, e0117133 (2015).
157. Yung, C. W., et al. Transglutaminase crosslinked gelatin as a tissue engineering scaffold. J. Biomed. Mater. Res. A 83A, 1039-1046 (2007).
158. Zhang, B. et al. Biodegradable scaffold with built-in vasculature for organ-on-a-chip engineering and direct surgical anastomosis. Nat. Mater. 15, 669-678 (2016).
159. Zhang, Q., et al. Thermal Transport in Quasi-1D van der Waals Crystal Ta2Pd3Se8 Nanowires: Size and Length Dependence. ACS Nano 12, 2634-2642 (2018).
160. Zhang, Y., et al. Characterization of printable cellular micro-fluidic channels for tissue engineering. Biofabrication 5, 025004 (2013).
161. Zhang, Y., et al. Thermal Transport in Electrospun Vinyl Polymer Nanofibers: Effects of Molecular Weight and Side Groups. Soft Matter (2018). doi: 10.1039/C8SM01696H
162. Zheng, Y., et al. In vitro microvessels for the study of angiogenesis and thrombosis. Proc. Natl. Acad. Sci. 109, 9342-9347 (2012).
163. Zhou, X. et al. Improved measurements of intracellular nitric oxide in intact microvessels using 4,5-diaminofluorescein diacetate. Am. J. Physiol.-Heart Circ. Physiol. 301, H108-H114 (2011).

What is claimed is:

1. A microvessel comprising:
(a) a microchannel formed in a hydrogel material; and
(b) a medial layer including a plurality of mural cells comprising smooth muscle cells, pericytes, or both smooth muscle cells and pericytes;
wherein each one of the plurality of mural cells is in contact with a wall of the microchannel; and
wherein the plurality of mural cells are helically aligned at an angle of from about 30 degrees to about 90 degrees relative to a lengthwise axis of the microchannel.

2. The microvessel of claim 1, wherein the plurality of smooth muscle cells are human brain, coronary, pulmonary, renal, skeletal muscle, hepatic, mesenteric, retinal, pancreatic, umbilical artery, umbilical vein microvascular smooth muscle cells, or any combination thereof.

3. The microvessel of claim 1, wherein the plurality of mural cells are arranged in a plurality of layers.

4. The microvessel of claim 3, further comprising a layer of coating material as a substrate for the plurality of mural cells.

5. The microvessel of claim 4, wherein the layer of coating material comprises collagen, fibronectin, vitronectin, laminin, elastin, cyclic RGD peptide, or any combination thereof.

6. The microvessel of claim 1, further comprising a layer of endothelial cells lining a lumen of the microvessel.

7. The microvessel of claim 1, wherein the microvessel has a stiffness of from about 0.1 to about 150 kPa.

8. A method for inducing vasoconstriction, vasodilation, or both in the microvessel of claim 1, the method comprising flowing a solution comprising at least one stimulus molecule through the microvessel.

9. The method of claim 8, wherein the at least one stimulus molecule comprises endothelin-1, U46619, HA-1077, or sodium nitroprusside.

10. A method for making a microvessel, the method comprising:
(a) fabricating a microchannel in a hydrogel material;
(b) seeding a plurality of mural cells comprising smooth muscle cells, pericytes, or both into the microchannel, wherein the smooth muscle cells adhere to a lumen wall of the microchannel;
(c) subjecting the plurality of mural cells to at least one treatment to align the plurality of mural cells helically, to cause the plurality of mural cells to exhibit a contractile phenotype, or both.

11. The method of claim 10, wherein the plurality of mural cells are seeded by flowing a suspension of the mural cells through a lumen of the microchannel.

12. The method of claim 10, wherein before step (b), a coating material comprising collagen, fibronectin, vitronectin, laminin, elastin, cyclic RGD peptide, or any combination thereof is flowed into the microchannel to coat the plurality of mural cells and step (b) is repeated prior to performing step (c).

13. The method of claim 10, wherein the at least one treatment helically aligns the plurality of mural cells at an angle of from about 30 degrees to about 90 degrees relative to a lengthwise axis of the microchannel.

14. The method of claim 10, wherein the plurality of smooth muscle cells are human brain, coronary, pulmonary, renal, skeletal muscle, hepatic, mesenteric, retinal, pancreatic, umbilical artery, umbilical vein microvascular smooth muscle cells, or any combination thereof.

15. The method of claim 10, further comprising applying a first layer of coating material over the plurality of mural cells.

16. The method of claim 15, wherein the layer of coating material comprises collagen, fibronectin, vitronectin, laminin, elastin, cyclic RGD peptide, or any combination thereof.

17. The method of claim 15, further comprising applying one or more additional layers of mural cells, coating material, or both, on the first layer of coating material.

18. The method of claim 10, further comprising:
(d) applying a plurality of endothelial cells to a lumen of the microvessel after performing step (c).

19. A freestanding microchannel comprising a lumen and a continuous wall surrounding the lumen, wherein the wall comprises an enzymatically crosslinked hydrogel material.

20. The freestanding microchannel of claim 19, wherein the microchannel further comprises a medial layer including a plurality of mural cells comprising smooth muscle cells, pericytes, or both smooth muscle cells and pericytes;
wherein each one of the plurality of mural cells is in contact with the wall of the microchannel; and
wherein the plurality of mural cells are helically aligned at an angle of from about 30 degrees to about 90 degrees relative to a lengthwise axis of the microchannel.
